(19) United States
(12) Patent Application Publication
(10) Pub. No.: US 2022/0406443 A1
(43) Pub. Date: Dec. 22, 2022

(54) DISPOSAL MANAGEMENT SYSTEM FOR MEDICAL DEVICES, MANAGEMENT SERVER, AND WASTE BIN MANAGEMENT METHOD

(71) Applicant: OLYMPUS CORPORATION, Tokyo (JP)

(72) Inventors: Haruna TADAKOSHI, Tokyo (JP); Takeo SUZUKI, Tokyo (JP)

(73) Assignee: OLYMPUS CORPORATION, Tokyo (JP)

(21) Appl. No.: 17/695,044

(22) Filed: Mar. 15, 2022

(30) Foreign Application Priority Data
Jun. 16, 2021 (JP) PCT/JP2021/022831

(51) Int. Cl. G16H 40/20; B65F 1/00; B65F 1/06; A61B 50/36

(52) U.S. Cl. CPC G16H 40/20; B65F 1/0006; B65F 1/06; A61B 50/36; B65F 2210/168; B65F 2210/152; B65F 2210/20; B65F 2210/128; A61B 1/00103

(57) ABSTRACT

A waste bin management unit manages identification information of a waste bin and identification information and a disposal date of the medical device that has been disposed of in the waste bin in association with each other. The waste bin management unit sets a scheduled date for collecting the waste bin based on at least either one of the number of medical devices that have been disposed of in the waste bin and the disposal date.

### BACKGROUND OF THE INVENTION

### 1. Field of the Invention

The present disclosure relates to a technology for managing waste bins for disposing of medical devices.

### 2. Description of the Related Art

In order to ensure medical safety and infection prevention, there are medical device products that are designed to be used only once. Such products are called single-use medical devices, and once those devices are used in a medical facility, the devices are disposed of in designated waste bins and collected by specialized business operators. In recent years, from the viewpoints of effective utilization of resources and reduction of medical waste, remanufacturing of used single-use medical devices has been attracting attention, in which the manufacturer/distributor is responsible for disassembling, cleaning, replacing parts, reassembling, sterilizing, etc., so that the devices can be used again.

Patent Literature 1 discloses a technology for managing endoscopes with statuses of “in examination use”, “used for examination”, “under cleaning”, “cleaned”, “in storage”, “in repair”, “on loan”, and “disposed”. The status of “disposed” indicates that the endoscope has been disposed of.

[Patent Literature 1] Japanese Patent Application Publication No. 2019-92994

### SUMMARY OF THE INVENTION

Used single-use medical devices need to be properly disposed of in waste bins in order to prevent accidental reuse by users. Further, since single-use medical devices are sterilized products packaged in sterile packs, single-use medical devices have a set use-by date, and expired single-use medical devices need to be properly disposed of due to having non-guaranteed quality. For this reason, it is desirable to establish a system to manage the disposal status of medical devices and the status of waste bins.

A disposal management system according to one aspect of the present disclosure includes: a waste bin in which a medical device is disposed of; and a waste bin management unit that manages identification information of the waste bin and identification information and a disposal date of the medical device that has been disposed of in the waste bin in association with each other. The waste bin management unit sets a scheduled date for collecting the waste bin based on at least either one of the number of medical devices that have been disposed of in the waste bin and the disposal date. The scheduled collection date in this case may include not only a date but also a time.

A disposal management system according to another aspect of the present disclosure includes: a waste bin in which a medical device is disposed of; and a waste bin management unit that manages identification information of the waste bin and identification information and a disposal date of the medical device that has been disposed of in the waste bin in association with each other. The waste bin management unit sets a collection schedule for the waste bin in reference to the type of the waste bin.

A management server according to yet another aspect of the present disclosure is a management server that manages a waste bin in which a medical device is disposed of, including: a waste bin management unit that manages identification information of the waste bin and identification information and a disposal date of the medical device that has been disposed of in the waste bin in association with each other. The waste bin management unit sets a scheduled date for collecting the waste bin based on at least either one of the number of medical devices that have been disposed of in the waste bin and the disposal date.

A waste bin management method according to yet another aspect of the present disclosure is a waste bin management method for managing a waste bin in which a medical device is disposed of, including: acquiring identification information and a disposal date of the medical device that has been disposed of in the waste bin; managing identification information of the waste bin and the identification information and the disposal date of the medical device that has been disposed of in the waste bin in association with each other; and setting a scheduled date for collecting the waste bin based on at least either one of the number of medical devices that have been disposed of in the waste bin and the disposal date.

A disposal management system according to still another aspect of the present disclosure includes: a first acquisition unit that has a function of acquiring scheduled disposal date information or use-by date information of the medical device associated with the identification information of the medical device; a second acquisition unit that has a function of acquiring disposal information indicating that the medical device having the identification information has been disposed of; and a disposal determination unit that determines whether or not the medical device has been disposed of by a scheduled disposal date or a use-by date based on the disposal information.

A disposal management system according to still another aspect of the present disclosure includes: a management unit that manages a scheduled disposal date or a use-by date of a medical device in association with identification information of one or more medical devices in a medical facility; an acquisition unit that acquires disposal information including identification information and disposal date information of a medical device that has been disposed of in the medical facility; and a disposal determination unit that identifies a medical device that has not been disposed of by a scheduled disposal date or a use-by date among the medical devices managed by the management unit based on the disposal information.

A management server according to still another aspect of the present disclosure is a management server capable of communicating with a facility system that acquires disposal information indicating that a medical device having identification information has been disposed of via the Internet, including: an acquisition unit that acquires scheduled disposal date information or use-by date information of the medical device associated with the identification information of the medical device; and a disposal determination unit that determines whether or not the medical device has been disposed of by a scheduled disposal date or a use-by date based on the disposal information.

A disposal management method according to still another aspect of the present disclosure includes: managing a scheduled disposal date or a use-by date of a medical device in association with identification information of one or more medical devices in a medical facility; acquiring disposal information including identification information and disposal date information of a medical device that has been disposed of in the medical facility; and identifying a medical device that has not been disposed of by a scheduled disposal date or a use-by date among the managed medical devices based on the disposal information.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of a disposal management system according to the first embodiment;

FIG. 2 is a diagram showing an exterior example of a waste bin;

FIG. 3 is a diagram showing functional blocks of the waste bin;

FIG. 4 is a diagram showing functional blocks of an endoscope management server;

FIG. 5 is a diagram for explaining information transmitted and/or received between a facility system and an external system;

FIG. 6 is a diagram showing an example of a management list recorded in a recording unit;

FIG. 7 is a diagram showing an example of a management list generated by a facility management unit;

FIG. 8 is a diagram showing an example of the management list recorded in the recording unit;

FIG. 9 is a diagram showing an example of the management list recorded in the recording unit;

FIG. 10 is a diagram showing a flowchart of a disposal determination process performed by a disposal determination unit;

FIG. 11 is a diagram showing an example of a disposal status determined by the disposal determination unit;

FIG. 12 is a diagram showing the configuration of a disposal management system according to the second embodiment;

FIG. 13 is a diagram for explaining information transmitted and/or received between a facility system and an external system;

FIG. 14 is a diagram showing an example of a management list generated by a facility management unit;

FIG. 15 is a diagram showing the configuration of a disposal management system according to the third embodiment;

FIG. 16 is a diagram showing functional blocks of the facility management unit;

FIG. 17 is a diagram for explaining information transmitted and/or received between the facility management unit and the waste bin;

FIG. 18 is a diagram showing an example of the management list recorded in the recording unit;

FIG. 19 is a diagram showing an example of the management list recorded in the recording unit;

FIG. 20 is a diagram showing an example of the disposal status determined by the disposal determination unit;

FIG. 21 is a diagram showing the configuration of the disposal management system according to the fourth embodiment;

FIG. 22A and FIG. 22B are diagrams showing an example of the type of the waste bin;

FIG. 23 is a diagram showing an example of the type of the waste bin;

FIG. 24 is a diagram showing functional blocks of the endoscope management server;

FIG. 25 is a diagram for explaining information transmitted and/or received between the facility system and the external system;

FIG. 26 is a diagram showing an example of the management list recorded in the recording unit;

FIG. 27 is a diagram showing an example of a scheduled collection date set by a waste bin management unit;

FIG. 28 is a diagram showing functional blocks of the waste bin;

FIG. 29 is a diagram showing a procedure for registering disposal information;

FIG. 30A shows an endoscopic examination vehicle, and

FIG. 30B shows an endoscope collection vehicle;

FIG. 31 is a diagram showing the configuration of a disposal management system according to the fifth embodiment;

FIG. 32 is a diagram showing functional blocks of the endoscope management server; and

FIG. 33 is a diagram showing an example of a management list with a field for a scheduled collection date and a field for a collection confirmation date.

### DETAILED DESCRIPTION OF THE INVENTION

The present disclosure relates to a technology for managing the disposal status of medical devices. In multiple embodiments shown below, medical devices subject to disposal management are single-use endoscopes. However, the medical devices may be any single-use medical devices other than endoscopes. Single-use endoscopes are packaged in sterile packs and transported from the manufacturing facility of the endoscopes to medical facilities in individual packaging boxes. The single-use endoscopes may be brought from the manufacturing facility to the medical facilities via a storage facility such as a warehouse. At a medical facility, single-use endoscopes are stored in a storage while being kept in individual packaging boxes, and when it is time to be used in an endoscopic examination, the single-use endoscopes are taken out from the storage and brought to an examination room. Since the single-use endoscopes are sterilized products, the expiration date is set to a date after a predetermined period from the date of manufacture, and expired single-use endoscopes must be disposed of immediately.

### First Embodiment

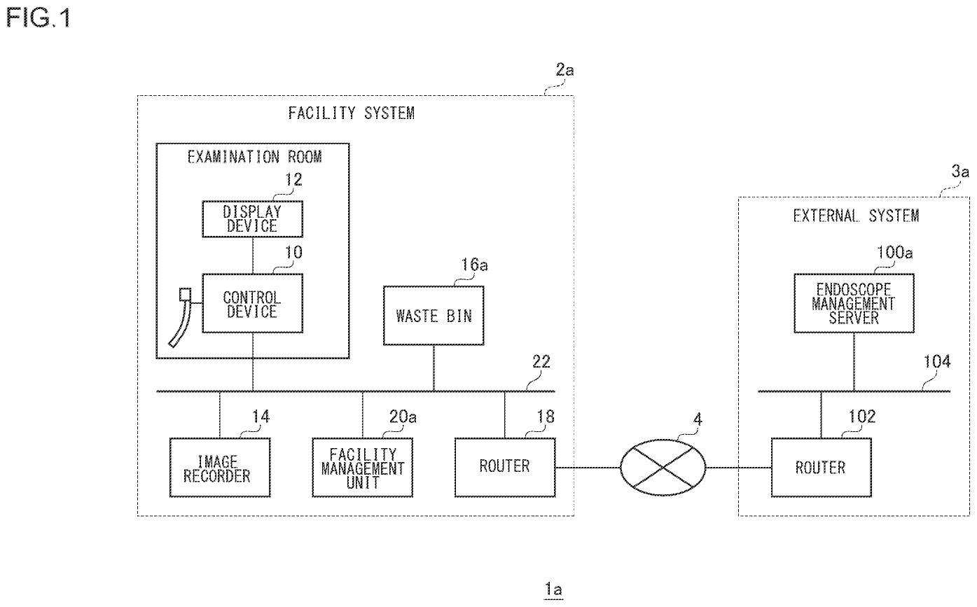

FIG. 1 shows the configuration of a disposal management system 1a in the first embodiment. The disposal management system 1a manages the disposal status of a single-use endoscope (hereinafter often simply referred to as “endoscope”) in a medical facility. The disposal management system 1a includes a facility system 2a, which is installed inside the medical facility, and an external system 3a, which is installed outside the medical facility. The facility system 2a and the external system 3a are communicably connected via Internet 4. The external system 3a may be operated by a medical device manufacturer that provides the endoscope to the medical facility.

The facility system 2a includes a control device 10, an image recorder 14, a waste bin 16a, and a facility management unit 20a. The control device 10, the image recorder 14, the waste bin 16a, and the facility management unit 20a are communicably connected by a network 22 such as a local area network (LAN). The network 22 is connected to the Internet 4 via a router 18.

The facility management unit 20a manages a work schedule in the medical facility. In the first embodiment, the facility management unit 20a manages a schedule for endoscopic examinations. However, the facility management unit 20a may also manage a schedule for medical operations such as other types of examinations and surgeries.

In a medical facility, an order for an endoscopic examination (hereinafter also referred to as “examination order”) is generated by an ordering system (not shown) and issued to the facility management unit 20a. The examination order includes order information related to the endoscopic examination such as the scheduled date and time of the start and end of the examination, patient identification information (patient ID), an examination type, a doctor in charge of the examination, and an examination room. Based on multiple examination orders issued by the ordering system, the facility management unit 20a sets an examination schedule in which multiple endoscopic examination dates are set.

Since the issued examination orders do not include information for identifying an endoscope to be used in the examinations, the facility management unit 20a assigns endoscopes that are available at the medical facility to the multiple endoscopic examinations included in the examination schedule. When the facility management unit 20a assigns an endoscope to an examination, the identification information of the endoscope assigned to the examination (hereinafter also referred to as “scope ID”) is registered in the examination schedule. Before the start of an endoscopic examination, a medical worker such as a nurse brings an endoscope with a scope ID registered in the examination schedule into the examination room and uses the endoscope in the examination.

There are various types of endoscopes corresponding to the type of the examination in this case such as otorhinolaryngological scopes, bronchoscopes, upper gastrointestinal general-purpose scopes, duodenal scopes, and colonoscopes. Therefore, the facility management unit 20a preferably assigns an endoscope to an examination that corresponds to the type of the endoscope. For example, an upper gastrointestinal general-purpose scope is assigned to a general examination for an upper part (routine examination), and a colonoscope is assigned to a colon examination. The assignment of a proper endoscope to an examination allows for efficient execution of the examination.

In the first embodiment, the facility management unit 20a assigns a single-use endoscope to an examination included in the examination schedule and determines a scheduled date (examination date) on which the single-use endoscope is to be used. The facility management unit 20a transmits scheduled date of use information indicating the determined scheduled date for use to the external system 3a along with the scope ID of the single-use endoscope. If the medical facility possesses a reusable endoscope that can be reused after cleaning, the facility management unit 20a may assign the reusable endoscope to the examination. However, the scheduled date of use information of the reusable endoscope does not need to be transmitted to the external system 3a.

Before the start of an endoscopic examination, the medical worker takes out an individual box of a single-use endoscope with a scope ID assigned to the examination from the storage and brings the individual packaging box to the examination room in reference to information registered in the examination schedule. For the identification of the single-use endoscope with the scope ID assigned to the examination, an identification target product pickup system (not shown) may be used. The medical worker opens the individual packaging box, takes out the endoscope packaged in a sterilization pack, then opens the sterilization pack to take out the endoscope, and connects the endoscope that has been taken out to the control device 10. The control device 10 has a function of controlling the functions of the endoscope.

During the examination, the control device 10 displays an image of the patient's internal body being captured by the endoscope on the display device 12, and the doctor observes each part of an organ that is displayed. When the doctor finds a lesion, the doctor operates a release switch of the endoscope so as to capture an image including the lesion. The control device 10 captures the image at the time when the release switch is operated and transmits the captured image to the image recorder 14. The control device 10 may transmit a plurality of captured images all at once to the image recorder 14 after the examination is completed.

The image recorder 14 is an image server that records images captured during an endoscopic examination and stores images transmitted from the control device 10 in association with an examination order. As metadata, the date and time of the image capturing, identification information of the endoscope (scope ID), information on the examination order, etc., may be added to a captured image.

The waste bin 16a is a disposal box with a box body in which an endoscope used in an examination (used endoscope) is disposed of, and is installed in the medical facility. The waste bin 16a is preferably installed near the examination room so that a medical worker can promptly dispose of a single-use endoscope used in an examination.

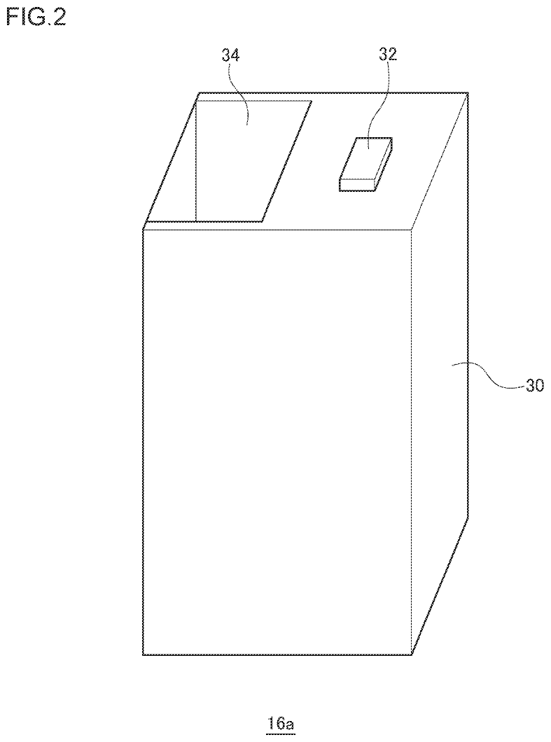

FIG. 2 shows an exterior example of the waste bin 16a. The waste bin 16a has a slot 34 for the insertion of a used endoscope, a box body 30 for receiving the inserted used endoscope, and an ID reader 32 for reading the identification information (scope ID) of the used endoscope. The slot 34 is provided with a flat lid that can be opened and closed, and the driving control of the lid may be performed such that the lid opens when the endoscope is disposed of and closes after the disposal of the endoscope.

The ID reader 32 has a function of reading the scope ID of an endoscope to be disposed of. The ID reader 32 may be an RFID reader that reads the scope ID from an RF tag attached to the endoscope. In the example shown in FIG. 2, the ID reader 32 is provided on the outside of the box body 30. Alternatively, the ID reader 32 may be provided on the inside of the box body 30.

The endoscope may hold the scope ID in the RF tag. Alternatively, for example, the endoscope may hold the scope ID in the form of a two-dimensional code such as a bar code or QR code (registered trademark). In either case, the endoscope holds the scope ID in a form that can be read by the ID reader 32, and a medical worker causes the ID reader 32 to read the scope ID of the endoscope to be disposed of.

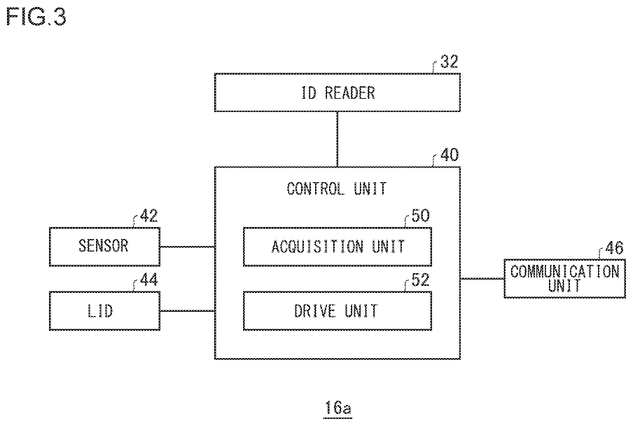

FIG. 3 shows functional blocks of the waste bin 16a. The waste bin 16a is formed having a control unit 40, an ID reader 32, a sensor 42, a lid 44, and a communication unit 46. The control unit 40 has an acquisition unit 50 that has a function of acquiring disposal information of an endoscope and a drive unit 52 that has a function of driving the lid 44.

The configuration shown in FIG. 3 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

Upon completing an endoscopic examination, the medical worker brings a used endoscope to the installation site of the waste bin 16a and causes the ID reader 32 to read the scope ID. Before the reading of the scope ID, the lid 44 is in a state where the slot 34 is closed, and when the ID reader 32 reads the scope ID, the drive unit 52 moves the lid 44 so as to open the slot 34. When a medical worker inserts a used endoscope in the box body 30 through the slot 34, the sensor 42 detects the insertion of the used endoscope, and the drive unit 52 moves the lid 44 so as to close the slot 34. The sensor 42 may be any sensor as long as the sensor can be installed in the box body 30 and can detect the insertion of an endoscope. For example, the sensor 42 may detect the insertion of an endoscope by detecting a change in the weight of items contained in the box body 30. The ID reader 32 may be installed at a position somewhat distant from the box body 30. Even in that case, when the ID reader 32 reads a scope ID, the read information may be transmitted to the control unit 40, and the drive unit 52 may move the lid 44 so as to open the slot 34. When the waste bin 16a is not provided with the lid 44, the ID reader 32 may be installed inside the box body 30 so as to automatically read the scope ID of an endoscope inserted into the box body 30 or may be installed near the slot 34 so as to automatically read the scope ID of an endoscope inserted into the box body 30.

When the ID reader 32 supplies the read scope ID to the control unit 40, the acquisition unit 50 acquires disposal information indicating that an endoscope having the scope ID has been disposed of. Upon receiving the scope ID, the acquisition unit 50 may generate disposal information or may acquire disposal information generated by another structure. After receiving the scope ID of the endoscope to be disposed of, the acquisition unit 50 may acquire disposal information indicating that an endoscope having the scope ID has been disposed of when the sensor 42 detects the insertion of the endoscope. The disposal information includes at least disposal date information indicating the date on which the endoscope was disposed of (disposal date). Alternatively, the disposal information may also include the date and time on which the endoscope was disposed of (disposal date and time). When the acquisition unit 50 acquires the disposal information, the communication unit 46 transmits the disposal information to the external system 3a. The communication unit 46 may transmit the disposal information to the facility management unit 20a.

The external system 3a includes an endoscope management server 100a that manages the disposal status of an endoscope. The endoscope management server 100a becomes connected to a network 104 such as a local area network (LAN) and becomes connected to the Internet 4 via a router 102. As described above, the external system 3a may be operated by a medical device manufacturer that provides the endoscope to the medical facility.

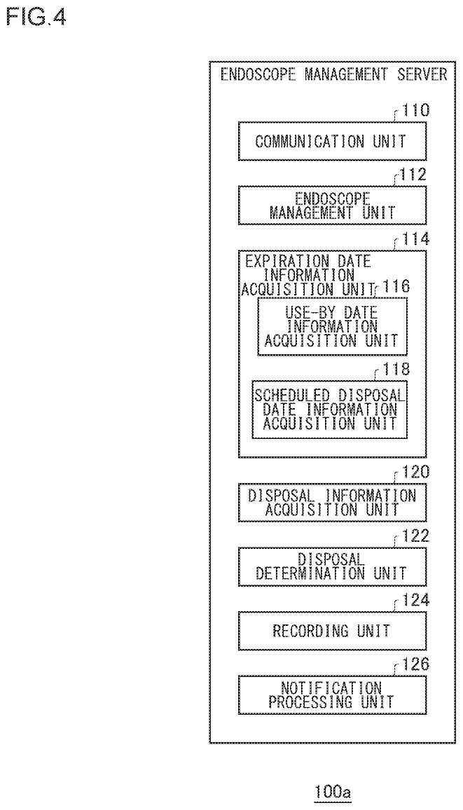

FIG. 4 shows functional blocks of the endoscope management server 100a. The endoscope management server 100a includes a communication unit 110, an endoscope management unit 112, an expiration date information acquisition unit 114, a disposal information acquisition unit 120, a disposal determination unit 122, a recording unit 124, and a notification processing unit 126. The expiration date information acquisition unit 114 has a function of acquiring scheduled disposal date information indicating the scheduled disposal date of an endoscope and/or use-by date information indicating the use-by date. In the first embodiment, the expiration date information acquisition unit 114 is formed having a use-by date information acquisition unit 116 and a scheduled disposal date information acquisition unit 118.

The configuration shown in FIG. 4 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

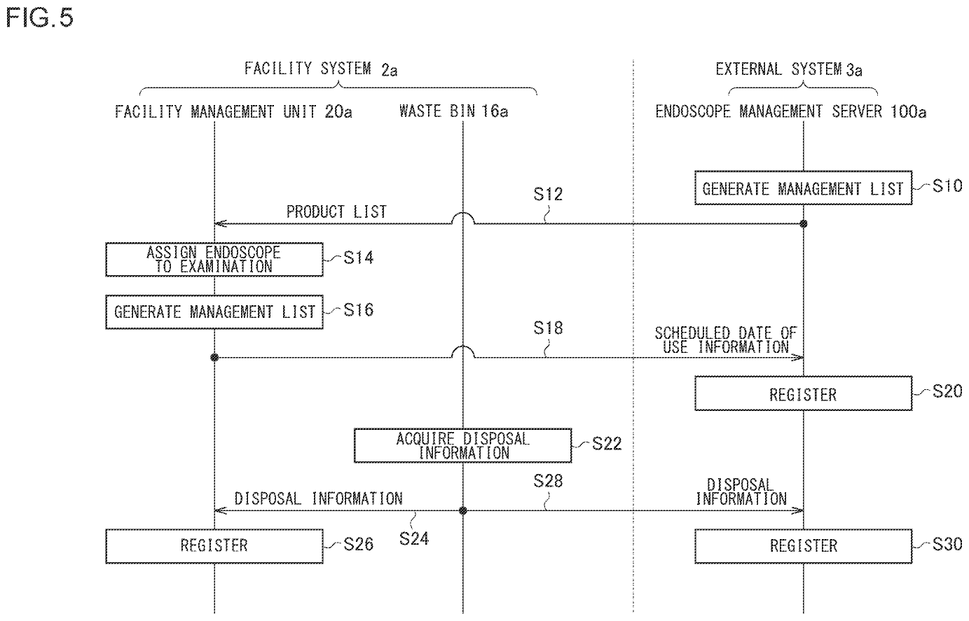

FIG. 5 is a diagram for explaining information transmitted and/or received between the facility system 2a and the external system 3a. In the first embodiment, the medical device manufacturer, which is the main operator of the endoscope management server 100a, manages the disposal status of single-use endoscopes provided to the medical facility. The endoscope management unit 112 generates a management list of endoscopes provided (delivered) to the medical facility and records the management list in the recording unit 124 (S10).

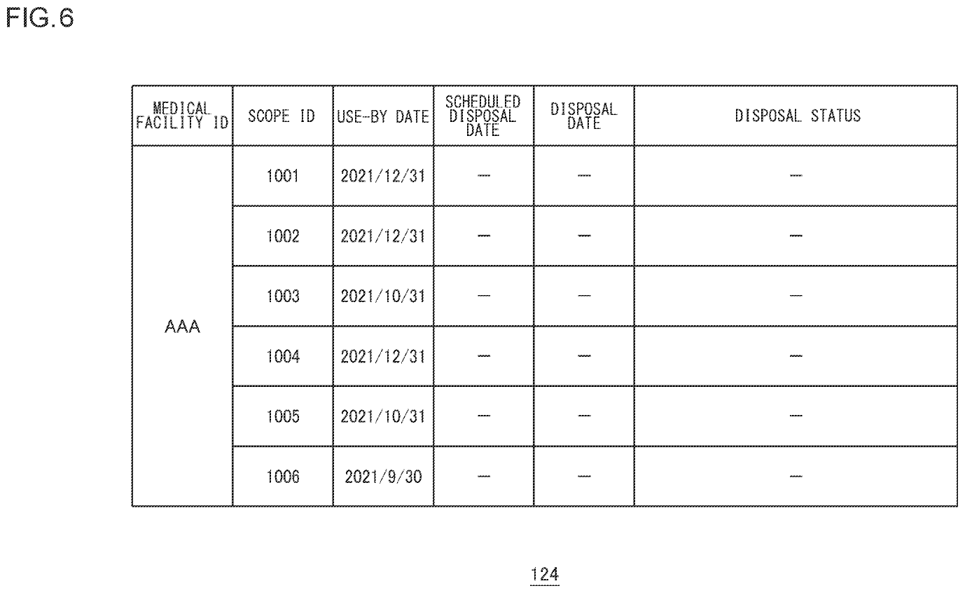

FIG. 6 shows an example of a management list recorded in the recording unit 124. This management list includes six single-use endoscopes provided to a medical facility with a medical facility ID “AAA”. FIG. 6 shows only some of endoscopes included in the management list, and in reality many endoscopes may be included.

The recording unit 124 holds the use-by date information of an endoscope in association with a scope ID. The use-by date information acquisition unit 116 acquires use-by date information by adding a predetermined effective period to the manufacturing date of each endoscope and provides the use-by date information associated with the scope ID to the endoscope management unit 112. If the effective period differs depending on the type of an endoscope, the use-by date information acquisition unit 116 acquires the use-by date information by using the effective period corresponding to the type of the endoscope. The endoscope management unit 112 registers the use-by date information acquired by the use-by date information acquisition unit 116 in the management list in association with the scope ID.

The endoscope management unit 112 transmits a list of endoscopes (product list) provided to the medical facility from the communication unit 110 to the facility system 2a (S12). The product list may include at least the scope ID of each endoscope and may further include the use-by date information of each endoscope.

In the facility system 2a, the facility management unit 20a receives the product list and extracts scope IDs included in the product list as the scope IDs of available endoscopes. If the product list is not transmitted from the external system 3a, the facility management unit 20a may acquire the scope IDs from products that actually arrive at the medical facility. The facility management unit 20a assigns an endoscope to an endoscopic examination corresponding to the type of the endoscope included in an examination schedule (S14) and determines the scheduled date of use of the endoscope. The facility management unit 20a may generate a management list in which the scheduled date of use is registered (S16).

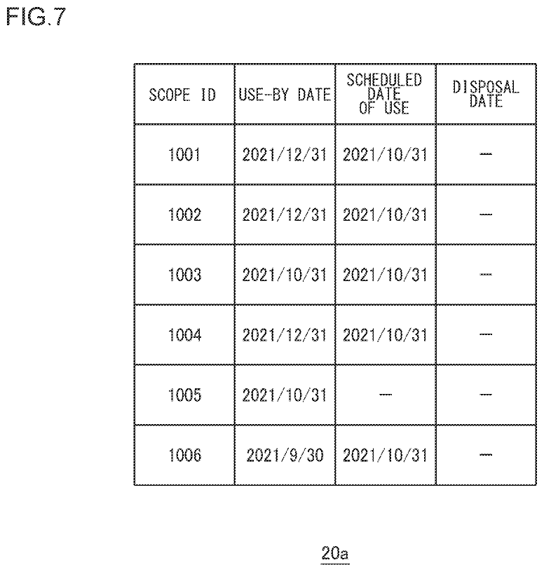

FIG. 7 shows an example of a management list generated by the facility management unit 20a. In the first embodiment, the endoscope management server 100a manages the disposal status of an endoscope. Therefore, the management list generated by the facility management unit 20a does not need to include fields for a use-by date and a disposal date.

Upon assigning an endoscope to an examination and determining the scheduled date of use of the endoscope, the facility management unit 20a associates scheduled date of use information indicating the determined scheduled date of use to the scope ID of the endoscope and then transmits the scheduled date of use information to the endoscope management server 100a (S18). In the endoscope management server 100a, the scheduled disposal date information acquisition unit 118 acquires the scheduled date of use information of the endoscope transmitted from the facility system 2a as the scheduled disposal date information of the endoscope. The first embodiment is premised on operation where a used endoscope is disposed of on the day the endoscope is used. However, if the endoscope is to be disposed of by the day after the day of use, the scheduled disposal date information acquisition unit 118 may acquire the next day of the scheduled date of use of the endoscope as the scheduled disposal date information of the endoscope. The scheduled disposal date information acquisition unit 118 provides the acquired scheduled disposal date information to the endoscope management unit 112 along with the scope ID. The endoscope management unit 112 registers the scheduled disposal date information acquired by the scheduled disposal date information acquisition unit 118 in the management list in association with the scope ID (S20).

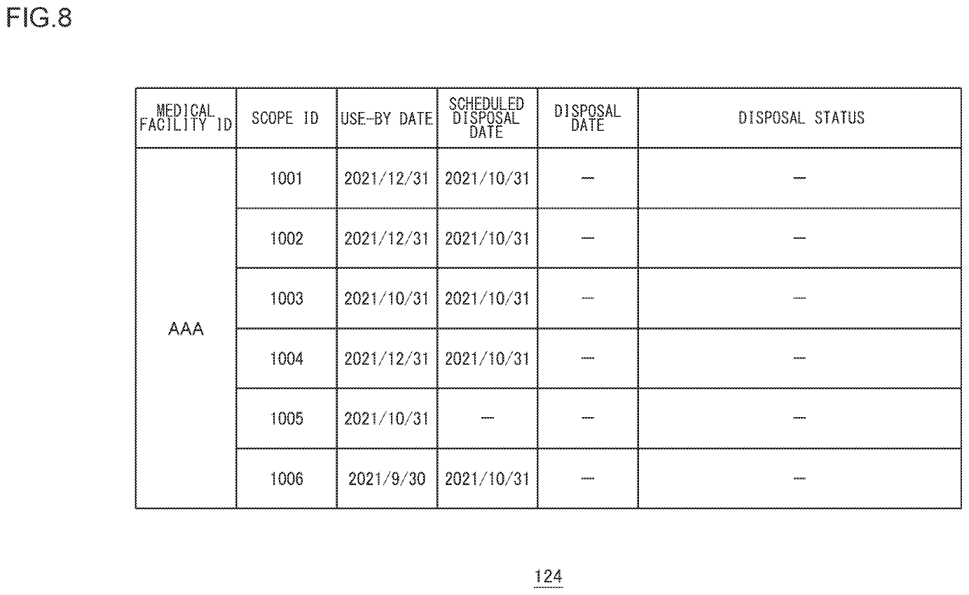

FIG. 8 shows an example of the management list recorded in the recording unit 124. The recording unit 124 holds the use-by date information and scheduled disposal date information of an endoscope in association with a scope ID. Since the use-by date indicates the last day on which the endoscope can be used, the scheduled disposal date (scheduled date of use) is not allowed to be set later than the use-by date. However, in the management list shown in FIG. 8, the scheduled disposal date (2021 Oct. 31) of an endoscope with a scope ID “1006” is set later than the use-by date (2021 Sep. 30). Therefore, the endoscope management unit 112 determines that the scheduled disposal date (2021 Oct. 31) of the endoscope with the scope ID “1006” is inappropriate, and the notification processing unit 126 transmits a message indicating that the scheduled date of use of the endoscope with the scope ID “1006” is inappropriate to the medical facility. In the facility system 2a, upon receiving the message from the endoscope management server 100a, the facility management unit 20a may inform the medical worker of the message.

In the management list shown in FIG. 8, the scheduled disposal date is not set for an endoscope with a scope ID “1005”. The possible reasons for this are that the endoscope with the scope ID “1005” has not been assigned to an examination or has been assigned to an examination but has not yet been registered in the facility system 2a. Upon registering that the endoscope with the scope ID “1005” has been assigned to an examination, the facility management unit 20a immediately transmits the scheduled date of use information of the endoscope with the scope ID “1005” to the endoscope management server 100a. The endoscope management unit 112 may update the management list every time the scheduled date of use information is transmitted from the facility management unit 20a. In this way, in association with the scope IDs of one or more endoscopes in a medical facility, the endoscope management unit 112 manages the use-by dates and/or the scheduled disposal dates of the endoscopes.

In the facility system 2a, when the waste bin 16a acquires disposal information indicating that an endoscope has been disposed of (S22), the waste bin 16a transmits the disposal information to the facility management unit 20a and the endoscope management server 100a (S24, S28). The disposal information includes at least the scope ID and the disposal date information. Upon receiving the disposal information, the facility management unit 20a registers the disposal date in a field for the disposal date of the scope ID (see FIG. 7) and updates the management list (S26). The facility management unit 20a may update the management list each time disposal information is transmitted from the waste bin 16a.

In the endoscope management server 100a, the disposal information acquisition unit 120 acquires disposal information transmitted from the waste bin 16a. The endoscope management unit 112 registers the disposal date in a field for the disposal date of the scope ID included in the disposal information (see FIG. 8) and updates the management list (S30). The endoscope management unit 112 may update the management list each time disposal information is transmitted from the waste bin 16a.

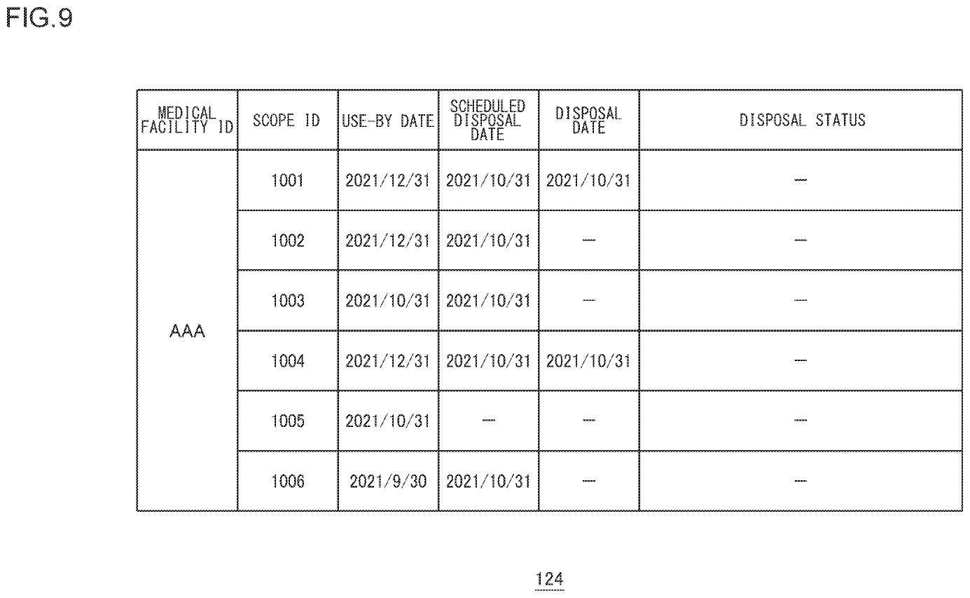

FIG. 9 shows an example of the management list recorded in the recording unit 124. The recording unit 124 holds the use-by date information, scheduled disposal date information, and disposal date information of an endoscope in association with a scope ID. In this example, the disposal information acquisition unit 120 has acquired the disposal information of endoscopes with the scope ID “1001” and the scope ID “1004” on Oct. 31, 2021, 1 and the endoscope management unit 112 has registered the disposal date in association with the scope IDs.

The disposal determination unit 122 determines whether or not an endoscope has been disposed of by the scheduled disposal date or the use-by date based on the disposal information. More specifically, the disposal determination unit 122 determines that the endoscope has not been disposed of by the scheduled disposal date or the use-by date if the disposal information has not been acquired by the disposal information acquisition unit 120 by the scheduled disposal date or the use-by date associated with the scope ID in the recording unit 124.

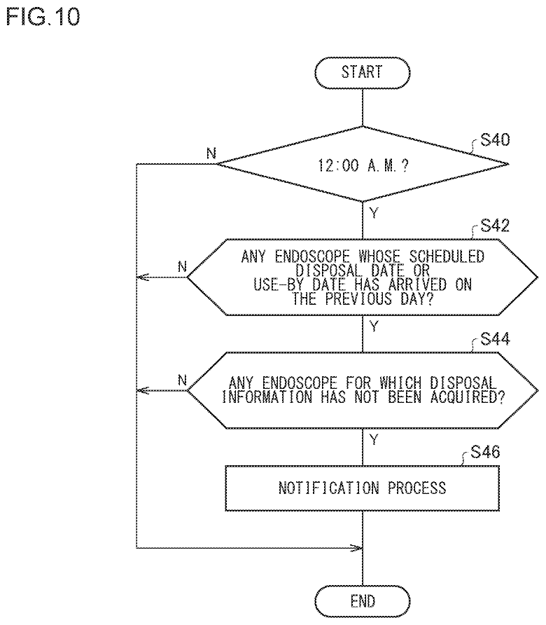

FIG. 10 shows a flowchart of a disposal determination process performed by the disposal determination unit 122. The disposal determination process is performed when the time reaches 12:00 a.m. (Y in S40) and is not performed at any other time (N in S40). In other words, the disposal determination process is performed every day at midnight when the date changes. The time at which the disposal determination process is performed may be set to be after the end of the work for the day and before the start of the work for the next day at the medical facility. In the following explanation, the time is assumed to be midnight on Nov. 1, 2021.

The disposal determination unit 122 determines whether there is an endoscope whose scheduled disposal date or use-by date associated with the scope ID has arrived on the previous day in reference to the management list recorded in the recording unit 124 (S42). If there is no endoscope whose scheduled disposal date or use-by date has arrived on the previous day (N in S42), the disposal determination process ends.

The previous day in this case is Oct. 31, 2021, and referring to the management list shown in FIG. 9, the scheduled disposal date or use-by date of endoscopes with scope IDs “1001”, “1002”, “1003”, “1004”, “1005”, and “1006” are set to Oct. 31, 2021. Therefore, the disposal determination unit 122 determines that the scheduled disposal date or use-by date of the endoscopes with the scope IDs “1001”, “1002”, “1003”, “1004”, “1005”, and “1006” has arrived on the previous day (Y in S42).

The disposal determination unit 122 checks whether there are any endoscopes for which disposal information has not been acquired among the endoscopes whose scheduled disposal date or use-by date has arrived on the previous day (S44). If disposal information has been acquired for all the endoscopes (N in S44), the disposal determination unit 122 determines that all the endoscopes have been properly disposed of, and the disposal determination process ends.

On the other hand, if there is an endoscope for which no disposal information has been acquired (Y in S44), the disposal determination unit 122 determines that the endoscope has not been properly disposed of by the scheduled disposal date or the use-by date. In the management list shown in FIG. 9, although the endoscopes with the scope IDs “1001” and “1004” have been disposed of by the scheduled disposal date or the use-by date, no disposal information has been acquired for the endoscopes with the scope IDs “1002”, “1003”, “1005”, and “1006”. Therefore, the disposal determination unit 122 determines that the endoscopes with the scope IDs “1002”, “1003”, “1005”, and “1006” have not been disposed of by the scheduled disposal date or the use-by date. As described, the disposal determination unit 122 according to the first embodiment identifies endoscopes that have not been disposed of by the scheduled disposal date or the use-by date among the endoscopes managed by the endoscope management unit 112 based on the disposal information.

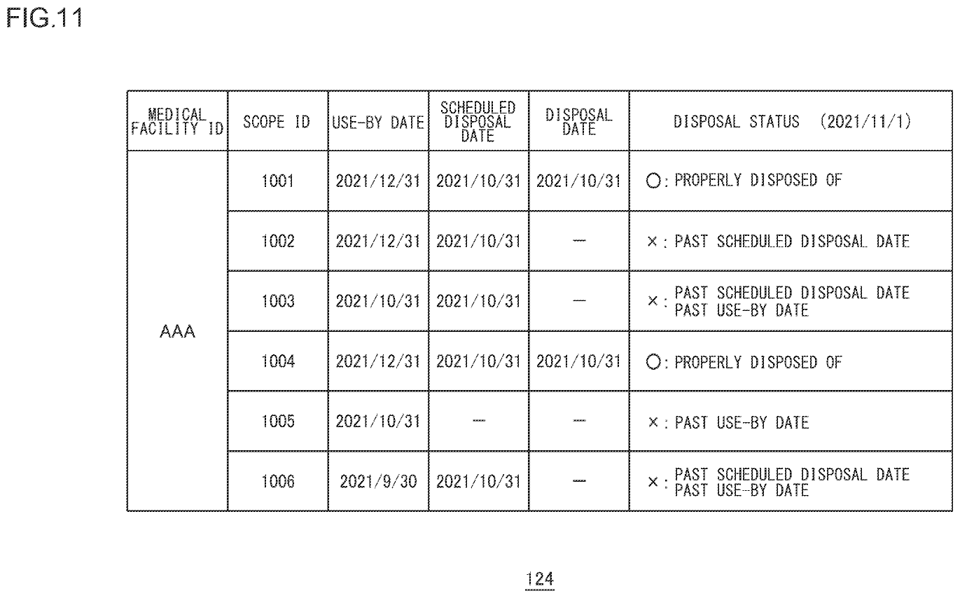

FIG. 11 shows an example of a disposal status determined by the disposal determination unit 122. The notification processing unit 126 may perform a notification process for the medical facility (S46). More specifically, the notification processing unit 126 transmits a message indicating that the endoscopes with the scope IDs “1002”, “1003”, “1005”, and “1006” have not been properly disposed of to the facility system 2a. In the facility system 2a, the facility management unit 20a informs the medical worker of the message, and the medical worker confirms that the endoscopes as informed have not been disposed of by the scheduled disposal date or the use-by date.

As described above, the scheduled disposal date (2021 Oct. 31) of the endoscope with the scope ID “1006” is set later than the use-by date (2021 Sep. 30), and it has already been determined that the scheduled disposal date is inappropriate by the time when the endoscope management unit 112 registers the scheduled disposal date. Therefore, the disposal determination unit 122 may exclude the endoscope with the scope ID “1006” from being a subject of the disposal determination process.

In the first embodiment in the above, the waste bin 16a transmits disposal information to the endoscope management server 100a (S28). Alternatively, after the disposal information is provided from the waste bin 16a to the facility management unit 20a, the facility management unit 20a may transmit the disposal information to the endoscope management server 100a.

In the first embodiment, the facility management unit 20a transmits scheduled date of use information to the endoscope management server 100a in association with a scope ID (S18). However, the scheduled date of use information does not need to be transmitted to the endoscope management server 100a. In this case, the endoscope management unit 112 may manage the disposal status of the endoscope based on the use-by date, and the endoscope management server 100a does not need to be able to communicate with the facility management unit 20a as long as the endoscope management server 100a can communicate with the waste bin 16a in the facility system 2a at least.

### Second Embodiment

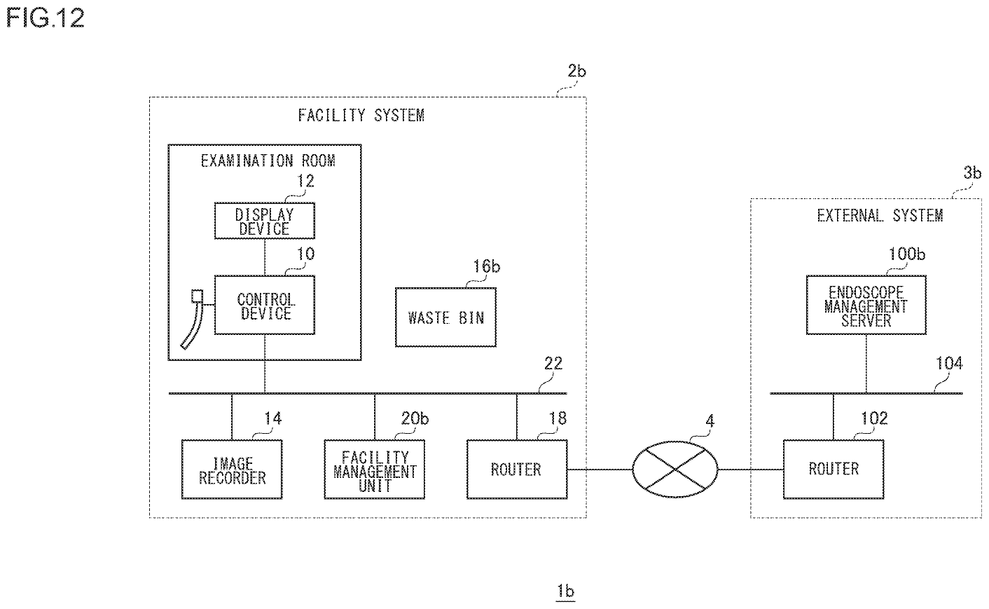

FIG. 12 shows the configuration of a disposal management system 1b in the second embodiment. The disposal management system 1b manages the disposal status of a single-use endoscope in a medical facility. The disposal management system 1b includes a facility system 2b, which is installed inside the medical facility, and an external system 3b, which is installed outside the medical facility. The facility system 2b and the external system 3b are communicably connected via the Internet 4. The external system 3b may be operated by a medical device manufacturer that provides the endoscope to the medical facility.

The facility system 2a includes a control device 10, an image recorder 14, a waste bin 16b, and a facility management unit 20b. The control device 10, the image recorder 14, and the facility management unit 20b are communicably connected by a network 22 such as a local area network (LAN). The network 22 is connected to the Internet 4 via a router 18.

The facility management unit 20b manages a work schedule in the medical facility. In the same way as in the facility management unit 20a in the first embodiment, the facility management unit 20b in the second embodiment sets an examination schedule in which the dates of multiple endoscopic examinations are set based on multiple examination orders issued by the ordering system and assigns endoscopes that are available at the medical facility to the multiple endoscopic examinations included in the examination schedule. Upon assigning a single-use endoscope to an examination included in the examination schedule and determining a scheduled date (examination date) on which the single-use endoscope is to be used, the facility management unit 20b transmits the scope ID of the single-use endoscope and scheduled date of use information indicating the determined scheduled date of use to the endoscope management server 100b.

The waste bin 16b has a box body in which an endoscope used in an examination (used endoscope) is disposed of and is installed in the medical facility. The waste bin 16b is preferably installed near the examination room so that a medical worker can promptly dispose of a single-use endoscope used in an examination.

Different from the waste bin 16a in the first embodiment, the waste bin 16b in the second embodiment is not provided with an ID reader 32 or a communication unit 46.

In other words, the waste bin 16b in the second embodiment does not have a function of reading the scope ID of an endoscope to be disposed of and does not have a function of transmitting disposal information, either.

The external system 3b includes an endoscope management server 100b that manages the disposal status of an endoscope. The endoscope management server 100b becomes connected to a network 104 such as a local area network (LAN) and becomes connected to the Internet 4 via a router 102. The endoscope management server 100b in the second embodiment is formed having the same or similar functions as those of the endoscope management server 100a in the first embodiment.

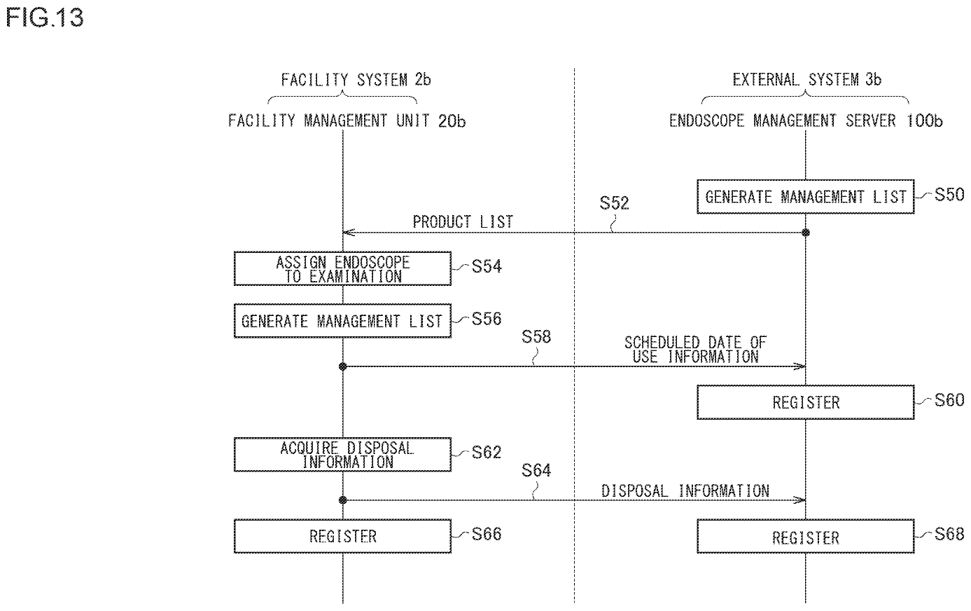

FIG. 13 is a diagram for explaining information transmitted and/or received between the facility system 2b and the external system 3b. In the second embodiment, the medical device manufacturer, which is the main operator of the endoscope management server 100b, manages the disposal status of single-use endoscopes provided to the medical facility. The endoscope management unit 112 generates a management list of endoscopes provided (delivered) to the medical facility and records the management list in the recording unit 124 (S50). An example of the management list recorded in the recording unit 124 is shown in FIG. 6.

The endoscope management unit 112 transmits a list of endoscopes (product list) provided to the medical facility from the communication unit 110 to the facility system 2b (S52). The product list may include at least the scope ID of each endoscope and may further include the use-by date information of each endoscope.

In the facility system 2b, the facility management unit 20b receives the product list and extracts scope IDs included in the product list as the scope IDs of available endoscopes. The facility management unit 20b assigns an endoscope to an endoscopic examination corresponding to the type of the endoscope included in an examination schedule (S54) and determines the scheduled date of use of the endoscope. The facility management unit 20b may generate a management list in which the scheduled date of use is registered (S56). An example of the management list generated by the facility management unit 20b is shown in FIG. 7.

Upon assigning an endoscope to an examination and determining the scheduled date of use of the endoscope, the facility management unit 20b associates scheduled date of use information indicating the determined scheduled date of use to the scope ID of the endoscope and then transmits the scheduled date of use information to the endoscope management server 100b (S58). In the endoscope management server 100b, the scheduled disposal date information acquisition unit 118 acquires the scheduled date of use information of the endoscope transmitted from the facility system 2b as the scheduled disposal date information of the endoscope. The scheduled disposal date information acquisition unit 118 provides the acquired scheduled disposal date information to the endoscope management unit 112 along with the scope ID. The endoscope management unit 112 registers the scheduled disposal date information acquired by the scheduled disposal date information acquisition unit 118 in the management list in association with the scope ID (S60). An example of the management list in which the scheduled disposal date information is registered is shown in FIG. 8.

In the medical facility, a medical worker disposes of a used endoscope in the waste bin 16b. At this time, the medical worker inputs the scope ID of the endoscope that has been disposed of to the facility management unit 20b. Upon receiving the input of the scope ID from the medical worker, the facility management unit 20b acquires disposal information indicating that the endoscope with the scope ID has been disposed of (S62). The disposal information includes at least disposal date information indicating the date on which the endoscope was disposed of (disposal date). Alternatively, the disposal information may also include disposal date information indicating the date and time on which the endoscope was disposed of (disposal date and time).

Upon acquiring disposal information indicating that an endoscope has been disposed of, the facility management unit 20b transmits the disposal information to the endoscope management server 100b (S64). In the endoscope management server 100b, the disposal information acquisition unit 120 acquires disposal information transmitted from the facility management unit 20b. The endoscope management unit 112 registers the disposal date in a field for the disposal date of the scope ID included in the disposal information (see FIG. 8) and updates the management list (S68). The endoscope management unit 112 may update the management list every time disposal information is transmitted from the facility management unit 20b. An example of the updated management list is shown in FIG. 9.

Upon acquiring the disposal information, the facility management unit 20b registers the disposal date in a field for the disposal date of the scope ID (see FIG. 7) and updates the management list (S66). The facility management unit 20b may update the management list each time disposal information is acquired.

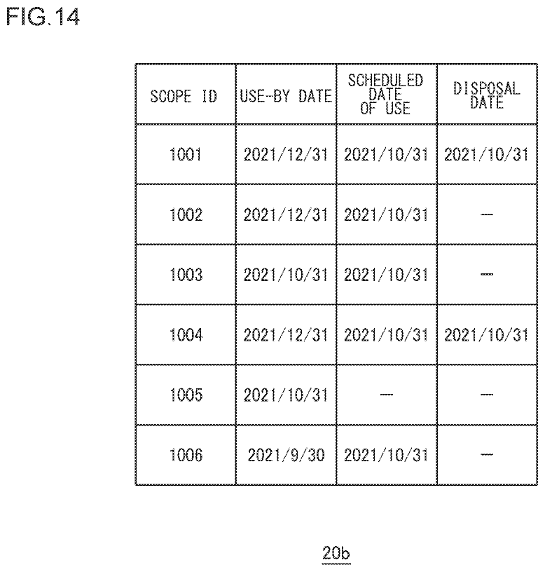

FIG. 14 shows an example of a management list generated by the facility management unit 20b. In the second embodiment, since the endoscope management server 100b manages the disposal status of an endoscope, the facility management unit 20b does not necessarily generate the management list.

In the second embodiment, the disposal determination unit 122 determines that the endoscope has not been disposed of by the scheduled disposal date or the use-by date if the disposal information has not been acquired by the scheduled disposal date or the use-by date associated with the scope ID in the recording unit 124. More specifically, the disposal determination unit 122 may perform the disposal determination process of a single-use endoscope according to the flowchart shown in FIG. 10.

### Third Embodiment

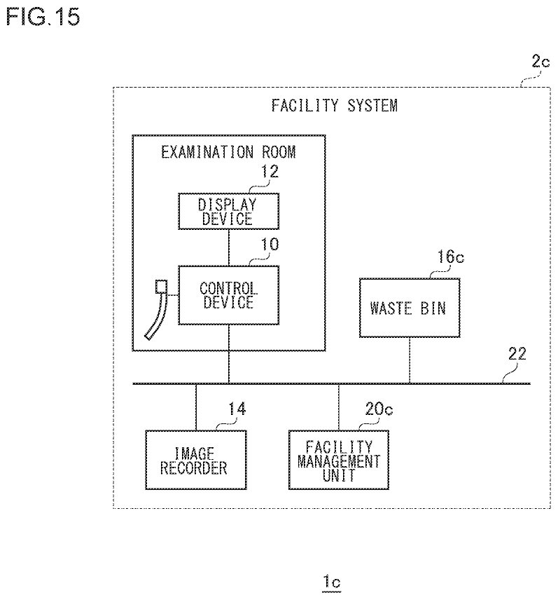

FIG. 15 shows the configuration of a disposal management system 1c in the third embodiment. The disposal management system 1c manages the disposal status of a single-use endoscope in a medical facility. The disposal management system 1c includes a facility system 2c provided in the medical facility.

The facility system 2c includes a control device 10, an image recorder 14, a waste bin 16c, and a facility management unit 20c. The control device 10, the image recorder 14, the waste bin 16c, and the facility management unit 20c are communicably connected by a network 22 such as a local area network (LAN).

The waste bin 16c has a box body in which an endoscope used in an examination (used endoscope) is disposed of and is installed in the medical facility. The waste bin 16c in the third embodiment is formed having the same or similar functions as those of the waste bin 16a in the first embodiment. In the third embodiment, the facility management unit 20c has a function of managing the disposal status of an endoscope.

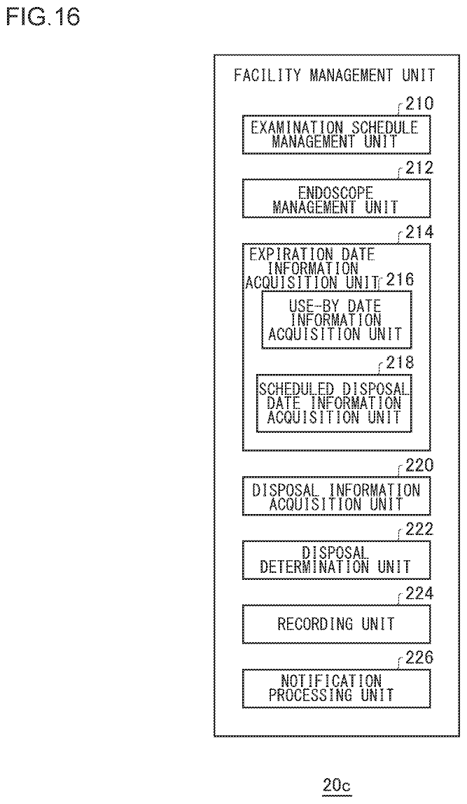

FIG. 16 shows functional blocks of the facility management unit 20c. The facility management unit 20c includes an examination schedule management unit 210, an endoscope management unit 212, an expiration date information acquisition unit 214, a disposal information acquisition unit 220, a disposal determination unit 222, a recording unit 224, and a notification processing unit 226. The expiration date information acquisition unit 214 has a function of acquiring scheduled disposal date information and/or use-by date information of an endoscope. In the third embodiment, the expiration date information acquisition unit 214 is formed having a use-by date information acquisition unit 216 and a scheduled disposal date information acquisition unit 218.

The configuration shown in FIG. 16 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The examination schedule management unit 210 manages a work schedule in the medical facility. In the third embodiment, the examination schedule management unit 210 manages a schedule for endoscopic examinations. However, the examination schedule management unit 210 may also manage a schedule for medical operations such as other types of examinations and surgeries. Based on multiple examination orders issued by the ordering system, the examination schedule management unit 210 sets an examination schedule in which the dates of multiple endoscopic examinations are set.

Since the issued examination orders do not include information for identifying an endoscope to be used in the examinations, the examination schedule management unit 210 assigns endoscopes that are available at the medical facility to the multiple endoscopic examinations included in the examination schedule. The assignment of an endoscope to an examination by the examination schedule management unit 210 causes the scope ID of the endoscope assigned to the examination to be registered in the examination schedule.

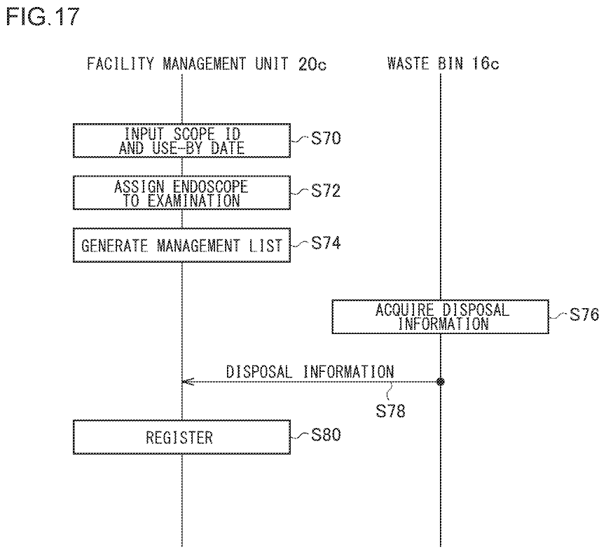

FIG. 17 is a diagram for explaining information transmitted and/or received between the facility management unit 20c and the waste bin 16c. The medical worker inputs to the facility management unit 20c the scope ID and use-by date of an endoscope that has been transported from the manufacturing facility of the endoscope and arrived at the medical facility (S70). The use-by date information acquisition unit 216 acquires the scope ID and the use-by date that have been input and provides the use-by date information associated with the scope ID to the endoscope management unit 212.

The examination schedule management unit 210 assigns an available endoscope to an endoscopic examination corresponding to the type of the endoscope included in an examination schedule (S72) and determines the scheduled date of use of the endoscope. The scheduled disposal date information acquisition unit 218 acquires the scheduled date of use information indicating the scheduled date of use of the endoscope determined by the examination schedule management unit 210 as the scheduled disposal date information of the endoscope. The scheduled disposal date information acquisition unit 218 provides the acquired scheduled disposal date information to the endoscope management unit 212 along with the scope ID. The endoscope management unit 212 generates a management list for managing the disposal status of the endoscope and records the management list in the recording unit 224 (S74).

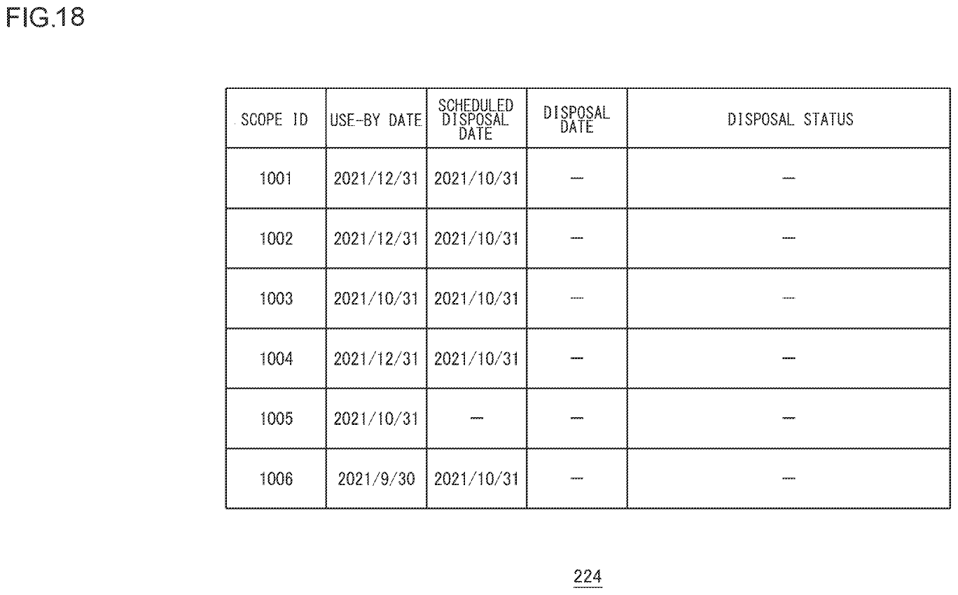

FIG. 18 shows an example of the management list recorded in the recording unit 224. The recording unit 224 holds the use-by date information and scheduled disposal date information of an endoscope in association with a scope ID. Since the use-by date indicates the last day on which the endoscope can be used, the scheduled disposal date (scheduled date of use) is not allowed to be set later than the use-by date. However, in the management list shown in FIG. 18, the scheduled disposal date (2021 Oct. 31) of an endoscope with a scope ID “1006” is set later than the use-by date (2021 Sep. 30). Therefore, the endoscope management unit 212 determines that the scheduled disposal date (2021 Oct. 31) of the endoscope with the scope ID “1006” is inappropriate, and the notification processing unit 226 may inform the medical worker of a message indicating that the scheduled date of use of the endoscope with the scope ID “1006” is inappropriate.

In the management list shown in FIG. 18, the scheduled disposal date is not set for an endoscope with a scope ID “1005”. The possible reasons for this are that the endoscope with the scope ID “1005” has not been assigned to an examination or has been assigned to an examination but has not yet been registered in the facility system 2c. When the examination schedule management unit 210 assigns the endoscope with the scope ID “1005” to an examination and registers the scope ID in the examination schedule, the scheduled disposal date information acquisition unit 218 acquires the scheduled disposal date information of the endoscope with the scope ID “1005” and provides the scheduled disposal date information to the endoscope management unit 212. The endoscope management unit 212 may update the management list each time scheduled disposal date information is provided from the scheduled disposal date information acquisition unit 218.

When the waste bin 16c acquires disposal information indicating that the endoscope has been disposed of (S76), the waste bin 16c transmits the disposal information to the facility management unit 20c (S78). The disposal information includes at least the scope ID and the disposal date information. In the facility management unit 20c, the disposal information acquisition unit 220 acquires the disposal information transmitted from the waste bin 16c. The endoscope management unit 212 registers the disposal date in a field for the disposal date of the scope ID included in the disposal information and updates the management list (S80). The endoscope management unit 212 may update the management list each time disposal information is transmitted from the waste bin 16c.

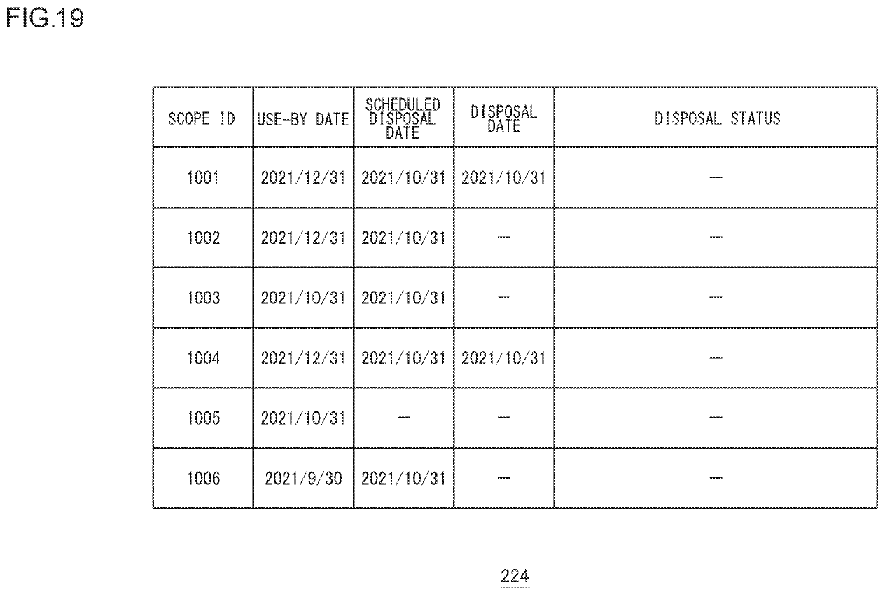

FIG. 19 shows an example of the management list recorded in the recording unit 224. The recording unit 224 holds the use-by date information, scheduled disposal date information, and disposal date information of an endoscope in association with a scope ID. In this example, the disposal information acquisition unit 220 acquires disposal information of the endoscopes with the scope IDs “1001” and “1004” on 2021 Oct. 31, and the endoscope management unit 212 resisters the disposal date in association with the both scope IDs.

The disposal determination unit 222 determines whether or not an endoscope has been disposed of by the scheduled disposal date or the use-by date based on the disposal information. More specifically, the disposal determination unit 222 determines that the endoscope has not been disposed of by the scheduled disposal date or the use-by date if the disposal information has not been acquired by the disposal information acquisition unit 220 by the scheduled disposal date or the use-by date associated with the scope ID in the recording unit 224. The disposal determination unit 222 may perform the disposal determination process of a single-use endoscope according to the flowchart shown in FIG. 10.

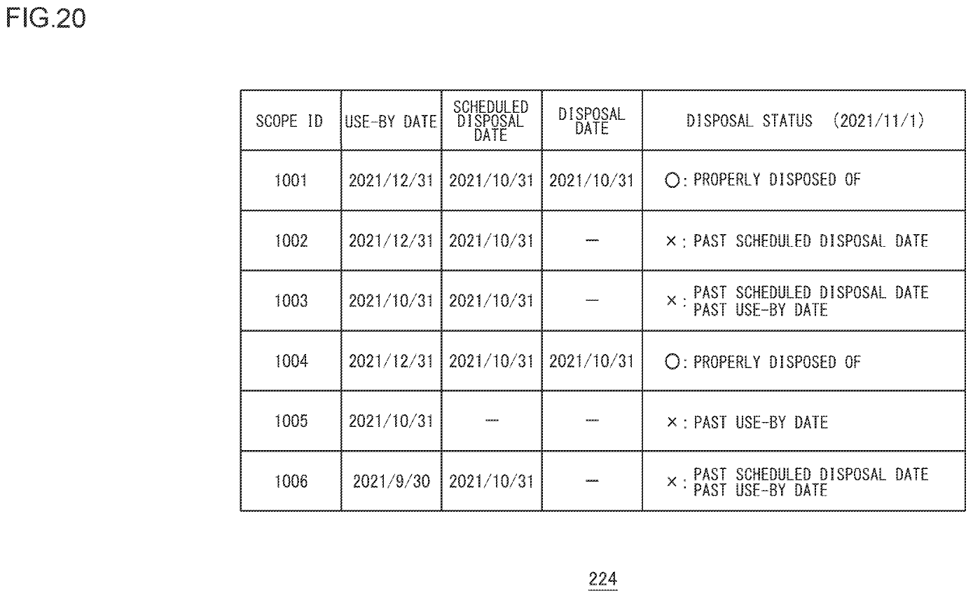

FIG. 20 shows an example of a disposal status determined by the disposal determination unit 222. The notification processing unit 226 may inform the medical worker of a message indicating that the endoscopes with the scope IDs “1002”, “1003”, “1005”, and “1006” have not been properly disposed of. This allows the medical worker to confirm that the endoscopes with the scope IDs as informed have not been disposed of by the scheduled disposal date or the use-by date.

In the third embodiment, the control device 10 may determine whether or not to restrict the function of the endoscope based on the use-by date of the endoscope. Prior to the start of the examination, when the endoscope is connected to the control device 10, the control device 10 acquires the scope ID from the endoscope. The control device 10 inquires the use-by date of the endoscope with the scope ID to the facility management unit 20c and acquires the use-by date information from the facility management unit 20c. At this time, if the current date is past the use-by date, the control device 10 determines to restrict the function of the endoscope. For example, the control device 10 may stop the power supply to the endoscope so as to disable the image capturing of the internal body of the patient. Alternatively, without restricting the image capturing by a camera, the control device 10 may stop the power supply to the display device 12 so that captured images cannot be displayed. The control device 10 may blur or partially hide a captured image displayed on the display device 12 or display a message on the captured image indicating that the endoscope cannot be used, thereby practically preventing the medical worker from using the endoscope. By restricting the function of the endoscope in this way, the control device 10 can practically prohibit the use of an expired single-use endoscope.

When use-by date information is stored in a recording unit provided in an endoscope in addition to the scope ID, the control device 10 acquires the scope ID and the use-by date information stored in the recording unit of the endoscope once the endoscope becomes connected to the control device 10. If the current date is past the use-by date, the control device 10 may determine to restrict the function of the endoscope.

In the third embodiment above, a medical worker inputs the scope ID and the use-by date of an endoscope to the facility management unit 20c (S70), and the endoscope management unit 212 registers the use-by date information in a management list in association with the scope ID (S74). The endoscope management unit 212 may update once registered use-by date information.

For example, if a sterilization pack of an endoscope is provided with an opening sensor for detecting the opening of the sterilization pack, the use-by date information acquisition unit 216 may set the use-by date and time to a predetermined time after the opening is detected by the opening sensor. The opening sensor may be a sensor that forms a conductive state with a conductor in the sterilization pack and detects a change in the conductive state due to the disconnection of the conductor when the pack is opened. Further, an opening sensor may be used that detects changes in the type or concentration of gas in the sterilization pack.

The sterilization pack has a function of transmitting the detection result to the facility management unit 20c once the opening sensor detects the opening. The use-by date information acquisition unit 216 sets the use-by date and time to a predetermined time after the time when the opening is detected, and the endoscope management unit 212 updates the use-by date and time in the management list. At this time, the disposal determination unit 222 monitors whether the disposal information of an endoscope with the scope ID has been acquired by the disposal information acquisition unit 220 by the updated use-by date and time. If the disposal information has not been acquired, the disposal determination unit 222 determines that the endoscope has not been disposed of by the use-by date and time.

### Fourth Embodiment

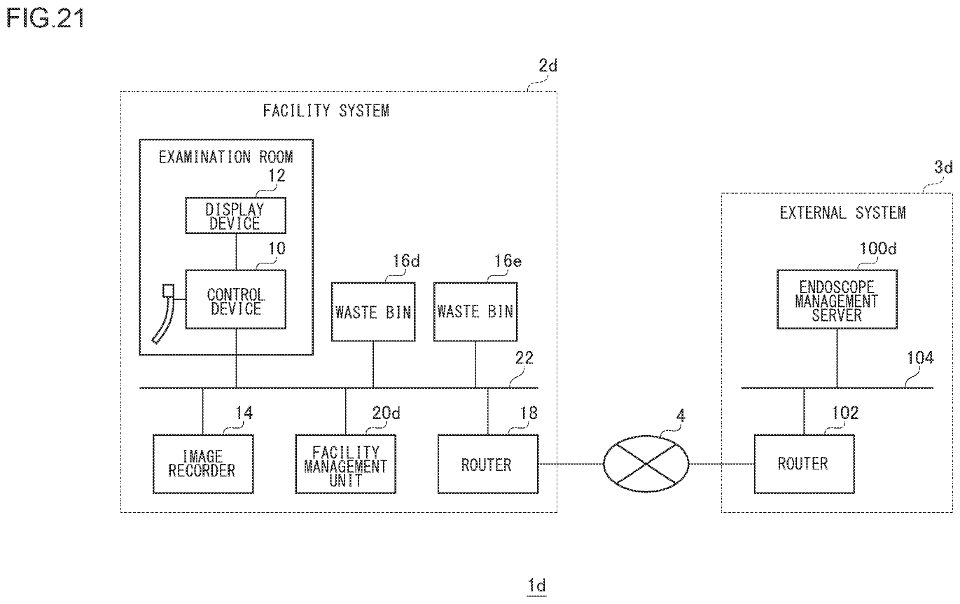

The fourth embodiment discloses a process of managing the status of a waste bin (waste box) and efficiently collecting the waste bin from a medical facility. FIG. 21 shows the configuration of a disposal management system 1d in the fourth embodiment. The disposal management system 1d manages the disposal status of a single-use endoscope in a medical facility and also manages the status of a waste bin, which is a waste box. The disposal management system 1d includes a facility system 2d, which is installed inside the medical facility, and an external system 3d, which is installed outside the medical facility. The facility system 2d and the external system 3d are communicably connected via Internet 4. The external system 3d may be operated by a medical device manufacturer that provides the endoscope to the medical facility.

The facility system 2d includes a control device 10, an image recorder 14, a waste bin 16d, a waste bin 16e, and a facility management unit 20d. The control device 10, the image recorder 14, the waste bin 16d, the waste bin 16e, and the facility management unit 20d are communicably connected by a network 22 such as a local area network (LAN). The network 22 is connected to the Internet 4 via a router 18.

The facility management unit 20d manages a work schedule in the medical facility. In the same way as in the facility management unit 20a in the first embodiment, the facility management unit 20d in the fourth embodiment sets an examination schedule in which the dates of multiple endoscopic examinations are set based on multiple examination orders issued by the ordering system and assigns endoscopes that are available at the medical facility to the multiple endoscopic examinations included in the examination schedule. Upon assigning a single-use endoscope to an examination included in the examination schedule and determining a scheduled date (examination date) on which the single-use endoscope is to be used, the facility management unit 20d transmits the scope ID of the single-use endoscope and scheduled date of use information indicating the determined scheduled date of use to the endoscope management server 100d.

The waste bins 16d and 16e have a box body in which an endoscope used in an examination (used endoscope) is disposed of and is installed in the medical facility. The waste bins 16d and 16e have identification information (waste bin ID), and the storing status of used endoscopes is managed by the endoscope management server 100d. The waste bins 16d and 16e in the fourth embodiment may be formed having the same or similar functions as those of the waste bin 16a in the first embodiment.

There is an upper limit to the number of endoscopes that can be stored in the respective box bodies of the waste bins 16d and 16e, and the waste bins 16d and 16e cannot store endoscopes beyond the upper limit. Therefore, the waste bins 16d and 16e are preferably collected promptly just before or when the number of endoscopes to be stored reaches the upper limit. The waste bins 16d and 16e are hereinafter simply referred to as “waste bin 16” if the waste bins 16d and 16e are not to be distinguished specifically.

The external system 3d includes an endoscope management server 100d that manages the disposal status of an endoscope and the collection schedule of a waste bin 16. The endoscope management server 100d becomes connected to a network 104 such as a local area network (LAN) and becomes connected to the Internet 4 via a router 102. The external system 3d may be operated by a medical device manufacturer that provides the endoscope and the waste bin 16 to the medical facility.

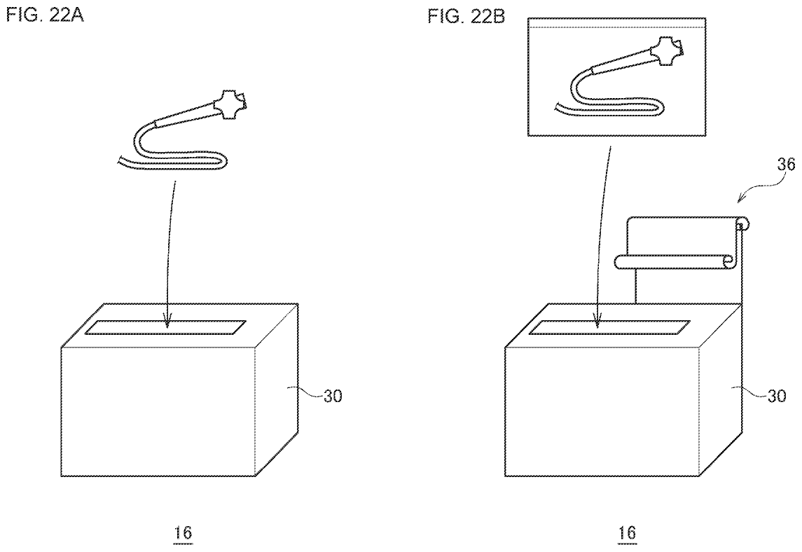

Various types of waste bins 16 may be installed in a medical facility. FIG. 22A shows an example of a type of waste bin 16 in which an endoscope is directly inserted. This type of waste bin 16 allows a medical worker to insert a used endoscope into a box body 30 without any trouble.

FIG. 22B shows an example of a type of waste bin 16 in which an endoscope put in a wrapping bag or a wrapping container is directly inserted. In this type of waste bin 16, a medical worker takes out a wrapping bag from a storage unit 36, puts a used endoscope in the wrapping bag, seals the wrapping bag, and inserts the sealed wrapping bag into a box body 30. Sealing the used endoscope in the wrapping bag makes the inside of the box body 30 less likely to get dirty. The wrapping bag may have a zipper, and the medical worker may put the used endoscope in the wrapping bag and close the zipper to seal the wrapping bag. Alternatively, a sealing device (sealer) for sealing a packaging bag may be installed in or near the box body 30, and the medical worker may use the sealing device to seal the wrapping bag. Note that a used endoscope may be put in a wrapping container for collection instead of a wrapping bag and then inserted in the box body 30.

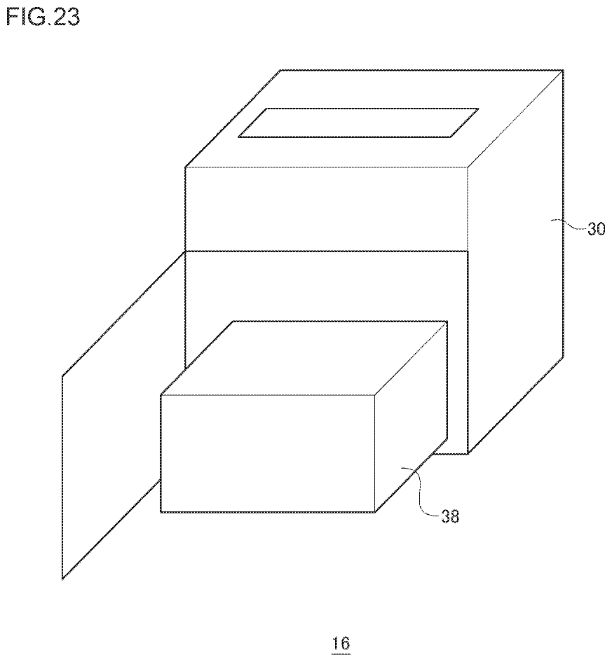

FIG. 23 shows a waste bin 16 of the type in which a collection bag or a collection container installed inside a box body 30 is collected. FIG. 23 shows a collection container 38 for collection installed inside the box body 30. Alternatively, a collection bag may be installed. The collection container 38 and the collection bag may be reusable or may be disposable. The collection container 38 and the collection bag may be foldable. In this type of waste bin 16, the box body 30 is permanently installed in a medical facility, and when used endoscopes accumulate in a collection container 38, the collection container 38 is taken out from the box body 30 so as to be collected.

There is also a type of waste bin 16 in which a collection container 38 is not installed inside thereof, and the entire box body 30 is collected. In particular, if the size of the box body 30 is not large, it is more advantageous from a sanitary standpoint to collect the entire box body 30. As described, there may be two types of waste bin 16: a type in which the entire box body 30 is collected; and a type in which a collection container 38 installed inside a box body 30 is collected. Further, the shape of the waste bin 16 can be various shapes such as a box, a bag, a pack, or a frame holding a bag, and it does not matter if there is a simple lid or not.

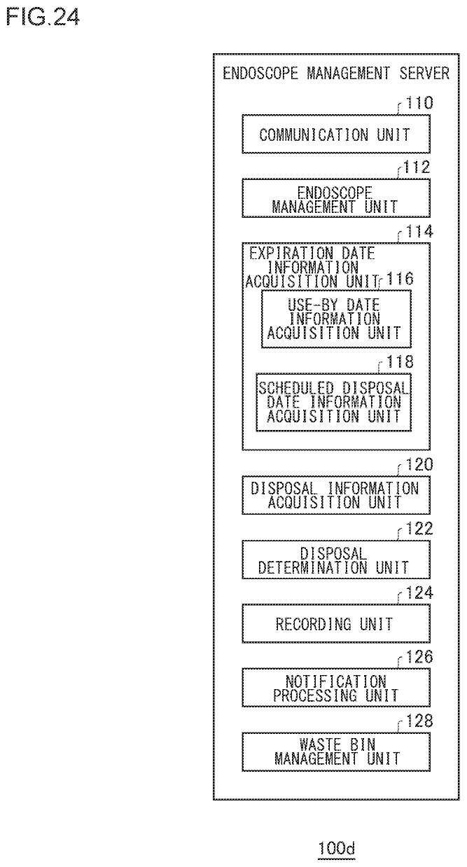

FIG. 24 shows functional blocks of the endoscope management server 100d. The endoscope management server 100d includes a communication unit 110, an endoscope management unit 112, an expiration date information acquisition unit 114, a disposal information acquisition unit 120, a disposal determination unit 122, a recording unit 124, a notification processing unit 126, and a waste bin management unit 128. The expiration date information acquisition unit 114 has a function of acquiring scheduled disposal date information and/or use-by date information of an endoscope. In the fourth embodiment, the expiration date information acquisition unit 114 is formed having a use-by date information acquisition unit 116 and a scheduled disposal date information acquisition unit 118.

The configuration shown in FIG. 24 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both. The endoscope management server 100d has a configuration obtained by adding a waste bin management unit 128 that manages the collection schedule of a waste bin to the configuration of the endoscope management server 100a in the first embodiment.

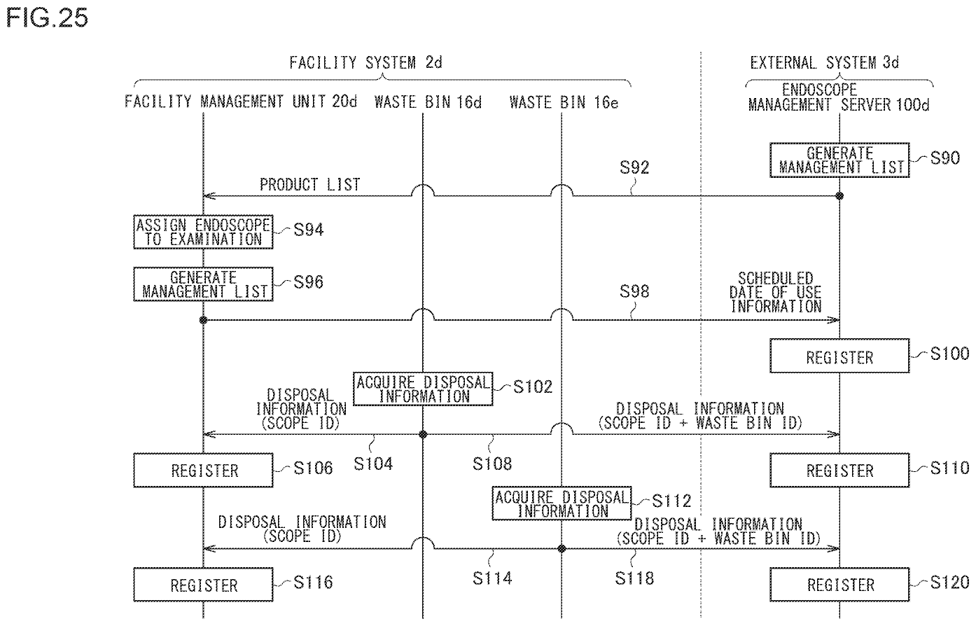

FIG. 25 is a diagram for explaining information transmitted and/or received between the facility system 2d and the external system 3d. In the fourth embodiment, the medical device manufacturer, which is the main operator of the endoscope management server 100d, manages the disposal status of single-use endoscopes provided to the medical facility and also manages the status of a waste bin. The endoscope management unit 112 generates a management list of endoscopes provided (delivered) to the medical facility and records the management list in the recording unit 124 (S90).

The endoscope management unit 112 transmits a list of endoscopes (product list) provided to the medical facility from the communication unit 110 to the facility system 2d (S92). The product list may include at least the scope ID of each endoscope and may further include the use-by date information of each endoscope.

In the facility system 2d, the facility management unit 20d receives the product list and extracts scope IDs included in the product list as the scope IDs of available endoscopes. The facility management unit 20d assigns an endoscope to an endoscopic examination corresponding to the type of the endoscope included in an examination schedule (S94) and determines the scheduled date of use of the endoscope. The facility management unit 20d may generate a management list in which the scheduled date of use is registered (S96).

Upon assigning an endoscope to an examination and determining the scheduled date of use of the endoscope, the facility management unit 20d associates scheduled date of use information indicating the determined scheduled date of use to the scope ID of the endoscope and then transmits the scheduled date of use information to the endoscope management server 100d (S98). In the endoscope management server 100d, the scheduled disposal date information acquisition unit 118 acquires the scheduled date of use information of the endoscope transmitted from the facility system 2d as the scheduled disposal date information of the endoscope. The scheduled disposal date information acquisition unit 118 provides the acquired scheduled disposal date information to the endoscope management unit 112 along with the scope ID. The endoscope management unit 112 registers the scheduled disposal date information acquired by the scheduled disposal date information acquisition unit 118 in the management list in association with the scope ID (S100). The endoscope management unit 112 updates the management list every time the scheduled date of use information is transmitted from the facility management unit 20d.

In the facility system 2d, when the waste bin 16d acquires disposal information indicating that an endoscope has been disposed of (S102), the waste bin 16d transmits the disposal information to the facility management unit 20d and the endoscope management server 100d (S104, S108). At this time, the disposal information transmitted to the facility management unit 20d includes the scope ID and the disposal date information, and the disposal information transmitted to the endoscope management server 100d includes the scope ID, the disposal date information, and a waste bin ID. Upon receiving the disposal information, the facility management unit 20d registers the disposal date information in association with the scope ID and updates the management list (S106). The facility management unit 20d may update the management list each time disposal information is transmitted from the waste bin 16d.

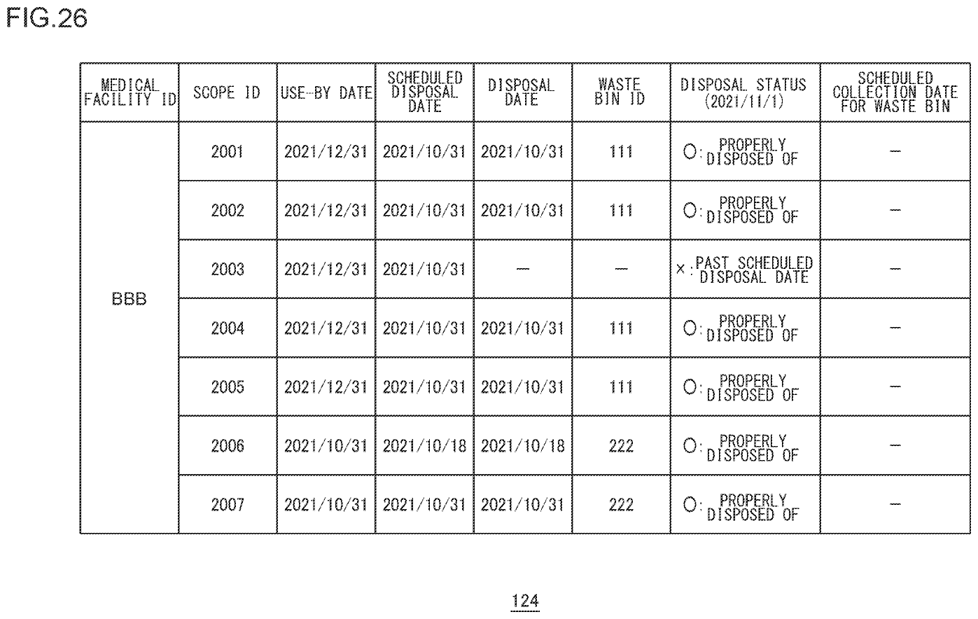

In the endoscope management server 100d, the disposal information acquisition unit 120 acquires disposal information transmitted from the waste bin 16d. The endoscope management unit 112 registers the disposal date in association with the scope ID included in the disposal information, and the waste bin management unit 128 registers the waste bin ID in association with the scope ID included in the disposal information and updates the management list (S110). The endoscope management unit 112 and the waste bin management unit 128 may update the management list each time disposal information is transmitted from the waste bin 16d. The management list according to the fourth embodiment will be explained in relation to FIG. 26.

When the waste bin 16e acquires disposal information indicating that an endoscope has been disposed of (S112), the waste bin 16e transmits the disposal information to the facility management unit 20d and the endoscope management server 100d (S114, S118). The disposal information transmitted to the facility management unit 20d includes the scope ID and the disposal date information, and the disposal information transmitted to the endoscope management server 100d includes the scope ID, the disposal date information, and a waste bin ID. Upon receiving the disposal information, the facility management unit 20d registers the disposal date in association with the scope ID and updates the management list (S116). The facility management unit 20d may update the management list each time disposal information is transmitted from the waste bin 16e.

In the endoscope management server 100d, the disposal information acquisition unit 120 acquires disposal information transmitted from the waste bin 16e. The endoscope management unit 112 registers the disposal date in association with the scope ID included in the disposal information, and the waste bin management unit 128 registers the waste bin ID in association with the scope ID included in the disposal information and updates the management list (S120). The endoscope management unit 112 and the waste bin management unit 128 may update the management list each time disposal information is transmitted from the waste bin 16e.

FIG. 26 shows an example of the management list recorded in the recording unit 124. This management list includes seven single-use endoscopes provided to a medical facility with a medical facility ID “BBB”. In the fourth embodiment, the waste bin 16d with a waste bin ID “111” and the waste bin 16e with a waste bin ID “222” are installed in the medical facility with the medical facility ID “BBB”. The disposal dates and the waste bin IDs are registered in the management list in association with the scope IDs of endoscopes that have been disposed of.

In the management list, the waste bin management unit 128 manages the waste bin IDs and the scope IDs and disposal dates of endoscopes disposed of in the waste bins in association with each other. In the management list shown in FIG. 26, endoscopes with scope IDs “2001”, “2002”, “2004”, and “2005” are disposed of in the waste bin 16d with the waste bin ID “111”.

For example, in a case where the upper limit number of endoscopes that can be disposed of in the waste bin 16d is five, collection efficiency is low if a collection plan is made after the disposal of the upper limit of five endoscopes since the waste bin 16d cannot be used until the waste bin 16d is actually collected. Therefore, upon detecting the disposal of a predetermined number (the upper limit number for collection) of endoscopes, which is less than the upper limit number for storage, in the waste bin 16d, the waste bin management unit 128 preferably recognizes the waste bin 16d as a collection target and sets a scheduled date for collecting the waste bin 16d.

In this example, the waste bin management unit 128 makes a collection plan when four endoscopes as compared to the upper limit of five endoscopes for storage are disposed of. Therefore, when the management list shown in FIG. 26 is generated, the waste bin management unit 128 determines that the waste bin 16d with the waste bin ID “111” is in a state that requires immediate collection and sets the scheduled collection date to 2021 Nov. 1, which is that very day. As described, the waste bin management unit 128 may set the scheduled date for collecting the waste bin 16d based on the number of endoscopes that have been disposed of in the waste bin 16d.

Since pathogens and microorganisms proliferate when used endoscopes are left unattended for a long period of time, it is not desirable for the endoscopes to be disposed of in the waste bin 16e and left unattended for a predetermined period of time or longer. Therefore, the waste bin management unit 128 may set the time when a predetermined period of time has passed since the disposal of the first used endoscope as the scheduled collection date for the waste bin 16e.

In this example, an endoscope with a scope ID “2006” has been disposed of in the waste bin 16e with the waste bin ID “222” on 2021 Oct. 18. Therefore, the waste bin management unit 128 may set the scheduled collection date to be a predetermined period of time (e.g., two weeks) after the date of the first disposal of an endoscope in the waste bin 16e so as to make a collection plan.

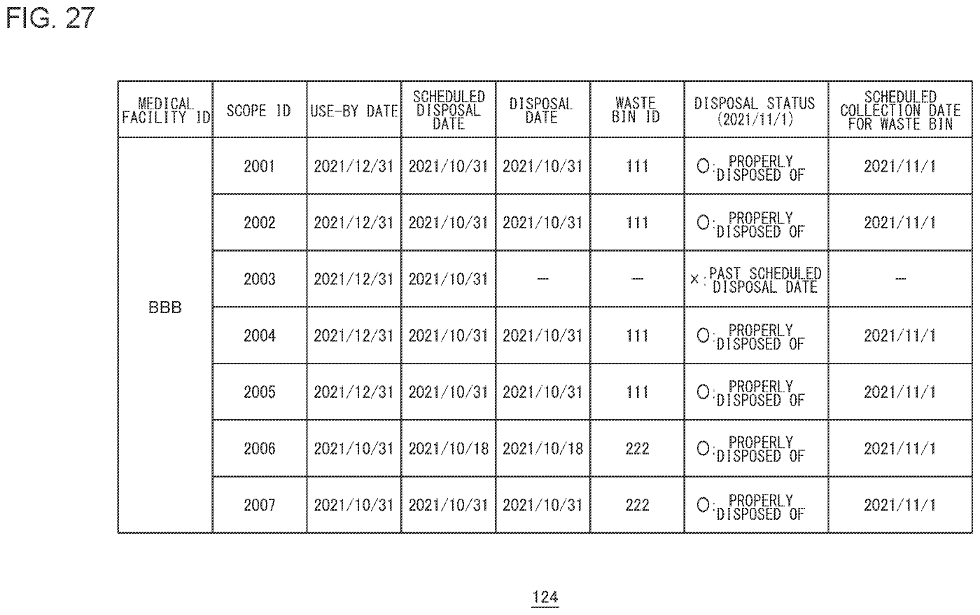

FIG. 27 shows an example of a scheduled collection date set by the waste bin management unit 128. The waste bin management unit 128 sets the scheduled collection date for the waste bin 16d (waste bin ID “111”) based on the number of endoscopes disposed of in the waste bin 16d and sets the scheduled collection date for the waste bin 16e (waste bin ID “222”) based on the disposal dates of the endoscopes. As described, the waste bin management unit 128 may set the scheduled date for collecting a waste bin based on at least either one of the number of endoscopes that have been disposed of in the waste bin and the disposal date.

The degree to which pathogens and microorganisms proliferate when a used endoscope is disposed of depends on the part of the body in which the endoscope was inserted. For example, endoscopes used in a digestive system such as large intestines and duodenums are more likely to have a higher number of pathogens attached to the endoscopes than endoscopes used in bronchial tubes and urinary organs.

Therefore, the upper limit for a collection period from the date of examination to the date of collection may be set for each type of examination, and a collection period upper limit for an endoscope used in a digestive system examination is set to be shorter than a collection period upper limit for an endoscope used in a bronchial or urological examination. Therefore, when setting a scheduled collection date for a waste bin based on a disposal date, the waste bin management unit 128 may refer to the type of an endoscope that has been disposed of, identify the upper limit for a collection period set for an examination corresponding to the type of the endoscope, and set the scheduled collection date for the waste bin in consideration of the upper limit for the collection period.

For example, the upper limit for the collection period for a bronchial or urological examination is set to 14 days, and the upper limit for the collection period for a gastrointestinal examination is set to five days. If an endoscope M that has been disposed of in a waste bin first was used for a bronchial examination, the waste bin management unit 128 sets the scheduled collection date for the waste bin to be two weeks after the disposal date. Then, if an endoscope N used for a colon examination is disposed of in the waste bin on the day after the disposal date of the endoscope M, the waste bin management unit 128 updates the scheduled collection date for the waste bin to be five days after the disposal date of the endoscope N. As described, the waste bin management unit 128 calculates the scheduled collection date for each endoscope in reference to the type of a used endoscope and sets the earliest date among calculated scheduled collection dates as the scheduled collection date for the waste bin. In this way, the collection of waste bins can be achieved in consideration of hygienic conditions.

Once the waste bin management unit 128 has set the scheduled collection date, the waste bin management unit 128 preferably reserves the schedule of a collector for collecting the waste bin on the scheduled collection date and the schedule of a carrier for delivering a replacement waste bin. For example, the waste bin management unit 128 may set a scheduled supply date for a new waste bin in reference to the scheduled date of use of an endoscope set in a work schedule at the medical facility. At this time, the waste bin management unit 128 preferably determines the type of the waste bin, for example, appropriate type and size (disposal capacity), based on the model and number of endoscopes to be used in a scheduled examination and reserves the schedule of the carrier such that the new waste bin can be supplied on the scheduled supply date.

When a used endoscope is disposed of in a waste bin, the degree to which the inside of the box body 30 becomes dirty depends on the type of waste bin. For example, comparing an direct-insertion type waste bin 16 shown in FIG. 22A and a type of waste bin 16 in which an endoscope put in a wrapping bag is inserted shown in FIG. 22B, the inside of a box body 30 is less likely to become dirty in the waste bin 16 of the type in which an endoscope put in a wrapping bag is inserted shown in FIG. 22B. Therefore, when setting a scheduled collection date for a waste bin based on a disposal date, the waste bin management unit 128 may set the scheduled collection date in reference to the type of the waste bin. For example, with regard to the waste bin 16 of the direct insertion type shown in FIG. 22A, the upper limit for the collection period for bronchial and urological examinations may be set to 10 days, and the upper limit for the collection period for gastrointestinal examinations may be set to 3 days. With regard to the waste bin 16 of the type in which an endoscope put in a wrapping bag is inserted shown in FIG. 22B, the upper limit for the collection period for bronchial and urological examinations may be set to 14 days, and the upper limit for the collection period for gastrointestinal examinations may be set to 5 days.

Further, the waste bin management unit 128 may set the scheduled collection date based on a disposal date depending on whether the type of the waste bin is a type in which the entire box is collected or a type in which a collection bag or a collection container installed inside the box is collected (see FIG. 23). The upper limit for the collection period may be set to be shorter for the waste bin 16 of the type in which a collection bag or a collection container is collected than for the waste bin 16 of the type in which the entire box is collected because the box itself is stationary in the former waste bin.

The waste bin management unit 128 may set the scheduled collection date based on the disposal date according to the type of the waste bin 16 such as the size and the shape. In this way, the waste bin management unit 128 can create an appropriate collection schedule according to the type of the waste bin 16 by setting the scheduled collection date for the waste bin 16 in reference to the type of the waste bin 16.

When the waste bin 16 is a type of waste bin in which an endoscope put in a wrapping bag or a wrapping container is inserted, the waste bin management unit 128 preferably sets a scheduled date for supplying wrapping bags or wrapping containers to be the same day as the scheduled collection date for the waste bin 16 or a day prior to the date on which the wrapping bags or the wrapping containers run out (for example, the day before the wrapping bags or the wrapping containers run out) based on the remaining amount of wrapping bags or wrapping containers. The waste bin management unit 128 manages the remaining amount of the wrapping bags or the wrapping containers (hereinafter simply referred to as “wrapping bags”). When the disposal information acquisition unit 120 acquires disposal information from a type of waste bin 16 in which an endoscope put in a wrapping bag is inserted, the waste bin management unit 128 determines that one wrapping bag has been used and reduces the remaining amount, which is managed by the waste bin management unit 128, by one. When the remaining amount of wrapping bags is estimated to be a predetermined number or less but not zero on the scheduled collection date for the waste bin 16, the waste bin management unit 128 sets the scheduled supply date for new wrapping bags to be the same day as the scheduled collection date for the waste bin 16. On the other hand, when the remaining amount of wrapping bags is estimated to be zero on the scheduled collection date for the waste bin 16, the waste bin management unit 128 sets the scheduled supply date for new wrapping bags before the remaining amount of the wrapping bags becomes zero. The process of estimating the remaining amount may be performed using the scheduled disposal date information for the endoscopes acquired by the scheduled disposal date information acquisition unit 118.

The waste bin management unit 128 may refer to temperature information of an environment in which the waste bin is located when setting the scheduled collection date for the waste bin based on the disposal date. When the temperature information of the environment shows relatively high temperature, the scheduled collection date may be set to a relatively early date, and when the temperature information of the environment shows relatively low temperature, the scheduled collection date may be set to a relatively late date. The temperature information of the environment may represent the average temperature for a predetermined period in the environment.

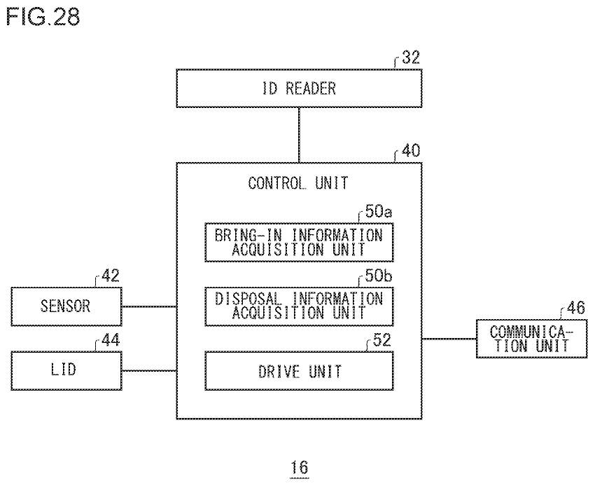

FIG. 28 shows functional blocks of a waste bin 16. The waste bin 16 is formed having a control unit 40, an ID reader 32, a sensor 42, a lid 44, and a communication unit 46. The control unit 40 has a bring-in information acquisition unit 50a that has a function of acquiring bring-in information of an endoscope, a disposal information acquisition unit 50b that has a function of acquiring disposal information of the endoscope, and a drive unit 52 that has a function of driving the lid 44.

The configuration shown in FIG. 28 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

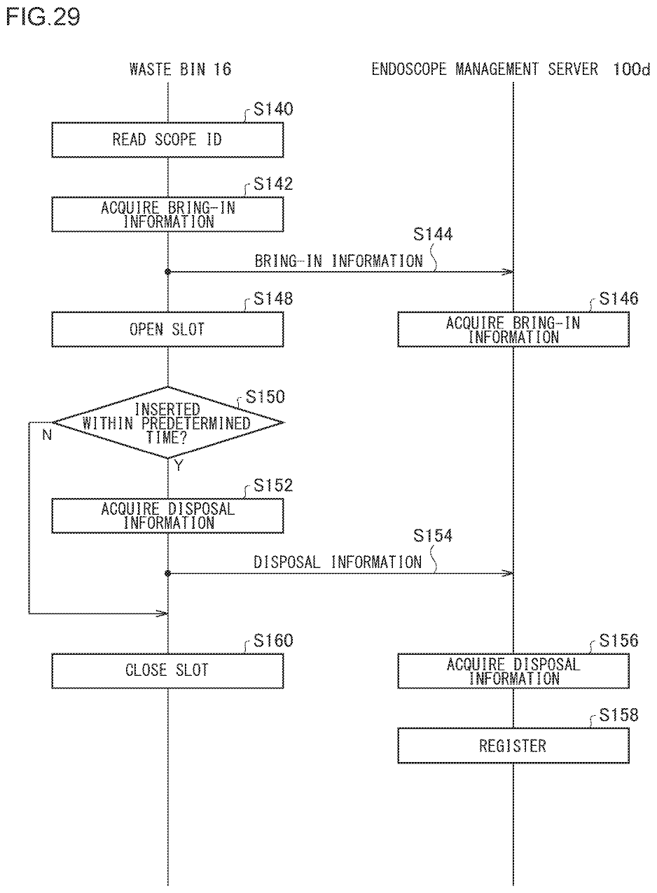

FIG. 29 shows an example of a procedure for registering disposal information. Upon completing an endoscopic examination, the medical worker brings a used endoscope in a disposal site at which a waste bin 16 is installed and causes the ID reader 32 (see FIG. 2) to read the scope ID. Before the reading of the scope ID, the lid 44 is in a state where the slot 34 is closed. When the ID reader 32 reads the scope ID (S140), the bring-in information acquisition unit 50a acquires bring-in information indicating that the endoscope has been brought in to the disposal site (S142). Since the bring-in information acquisition unit 50a acquires the bring-in information on the condition that the ID reader 32 has read the scope ID, the scope ID of the endoscope to be disposed of can be properly identified. The communication unit 46 transmits the bring-in information to the endoscope management server 100d (S144), and the disposal information acquisition unit 120 acquires the bring-in information in the endoscope management server 100d (S146).

When the bring-in information acquisition unit 50a acquires the bring-in information, the drive unit 52 moves the lid 44 so as to open the slot 34 (S148). As described, the acquisition of the bring-in information by the bring-in information acquisition unit 50a causes the slot 34 to be opened and the waste bin 16 to be in a state where the endoscope can be disposed of. When a medical worker inserts a used endoscope into the box body 30 through the slot 34 within a predetermined time (e.g., three minutes) after the slot 34 is opened (Y in S150), the sensor 42 detects the insertion of the used endoscope, and the disposal information acquisition unit 50b acquires disposal information indicating that the endoscope has been disposed of in the waste bin 16 (S152). The disposal information may include the scope ID, the disposal date information, and the waste bin ID. However, when the scope ID is included in the transmitted bring-in information, the scope ID does not need to be included. The sensor 42 may be any sensor as long as the sensor can be installed in the box body 30 and can detect the insertion of an endoscope. For example, the sensor 42 may detect the insertion of an endoscope by detecting a change in the weight of items contained in the box body 30.

The communication unit 46 transmits the disposal information to the endoscope management server 100d (S154), and the disposal information acquisition unit 120 acquires the disposal information in the endoscope management server 100d (S156). When the disposal information acquisition unit 120 acquires both the bring-in information and the disposal information, the endoscope management unit 112 registers the disposal date in association with the scope ID included in the disposal information or the bring-in information (S158). At this time, the waste bin management unit 128 may register the waste bin ID in association with the scope ID included in the disposal information or bring-in information and update the management list. When the disposal information acquisition unit 50b acquires the disposal information, the drive unit 52 moves the lid 44 so as to close the slot (S160).

When the medical worker does not insert the used endoscope into the box body 30 through the slot 34 within a predetermined time after the slot 34 is opened (N in S150), the drive unit 52 moves the lid 44 so as to close the slot 34 (S160). At this time, since the disposal information acquisition unit 50b does not acquire the disposal information, the disposal information is not transmitted to the endoscope management server 100d, and the endoscope management unit 112 never registers the disposal date. As described, preventing the disposal information from being transmitted to the endoscope management server 100d if an endoscope is not inserted through the slot 34 even when the ID reader 32 reads the scope ID and the slot 34 is opened allows for proper management of the status of the waste bin 16 and the endoscope. In the endoscope management server 100d, if the disposal information is not acquired within a predetermined time after the disposal information acquisition unit 120 acquires the bring-in information, the acquired bring-in information may be discarded.

In the example shown in FIG. 29, when the ID reader 32 reads the scope ID, the bring-in information acquisition unit 50a acquires the bring-in information. Alternatively, the bring-in information acquisition unit 50a may acquire the bring-in information when the ID reader 32 reads different information. For example, when a used endoscope is put in a wrapping bag and inserted into the waste bin 16, a bag ID attached to the wrapping bag that is for identifying the wrapping bag may be read by the ID reader 32, thereby causing the bring-in information acquisition unit 50a to acquire the bring-in information.

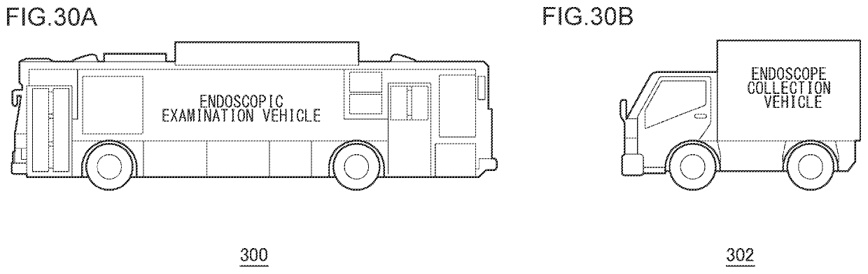

FIG. 30A shows an example of an endoscopic examination vehicle. An endoscopic examination vehicle 300 includes one or more endoscopic examination rooms in the vehicle. In the endoscopic examination room provided in the endoscopic examination vehicle 300, a control device 10 that controls the function of an endoscope and a display device 12 are arranged. During the examination, the control device 10 displays an image of the patient's internal body being captured by the endoscope on the display device 12, and the doctor observes each part of an organ that is displayed. The endoscopic examination vehicle 300 may be equipped with the structure of the facility system 2d shown in FIG. 21.

As described above, the endoscope management server 100d may manage the status of a waste bin 16 mounted on the endoscopic examination vehicle 300 and set a collection schedule for efficiently collecting the waste bin 16 from the endoscopic examination vehicle 300. For example, when the upper limit of the number of endoscopes that can be disposed of in the waste bin 16 is set, the waste bin management unit 128 may recognizes the waste bin 16 as a collection target and set a collection schedule including a scheduled time for collecting the waste bin 16 upon detecting the disposal of a predetermined number (the upper limit number for collection) of endoscopes, which is less than the upper limit number for storage, in the waste bin 16.

FIG. 30B shows an example of an endoscope collection vehicle. An endoscope collection vehicle 302 moves to a position where the endoscopic examination vehicle 300 is parked at the scheduled collection time according to the collection schedule set by the waste bin management unit 128. At this time, the endoscopic examination vehicle 300 may be loaded with a new waste bin 16 for replacement. The endoscope collection vehicle 302 may be a self-driving car or may be driven by a worker of the collector. The endoscope collection vehicle 302 may load the waste bin 16 collected from the endoscopic examination vehicle 300 and transports the waste bin 16 to a processing factory according to the collection schedule.

The waste bin management unit 128 may set the collection schedule for the waste bin 16 in reference to the type of the waste bin 16 mounted on the endoscopic examination vehicle 300. In this case, the type of the waste bin 16 includes the type and the size (disposal capacity), and the waste bin management unit 128 sets the optimal collection schedule according to the type of the waste bin 16. The collection schedule may include the transportation to the processing factory for used endoscopes after the endoscope collection vehicle 302 collects the waste bin 16 from the endoscopic examination vehicle 300.

Since the endoscopic examination vehicle 300 can self-travel, the waste bin management unit 128 may set a collection schedule for the endoscopic examination vehicle 300 to travel to the processing factory and collect the waste bin 16. In this way, the waste bin management unit 128 can set a collection schedule suitable for the type of the waste bin 16 by referring to the type of the waste bin 16. In this example, it is explained that the waste bin 16 is placed in the endoscopic examination vehicle 300. Alternatively, the waste bin 16 may be placed in the endoscope collection vehicle 302 for collecting endoscopes used in the endoscopic examination vehicle 300.

### Fifth Embodiment

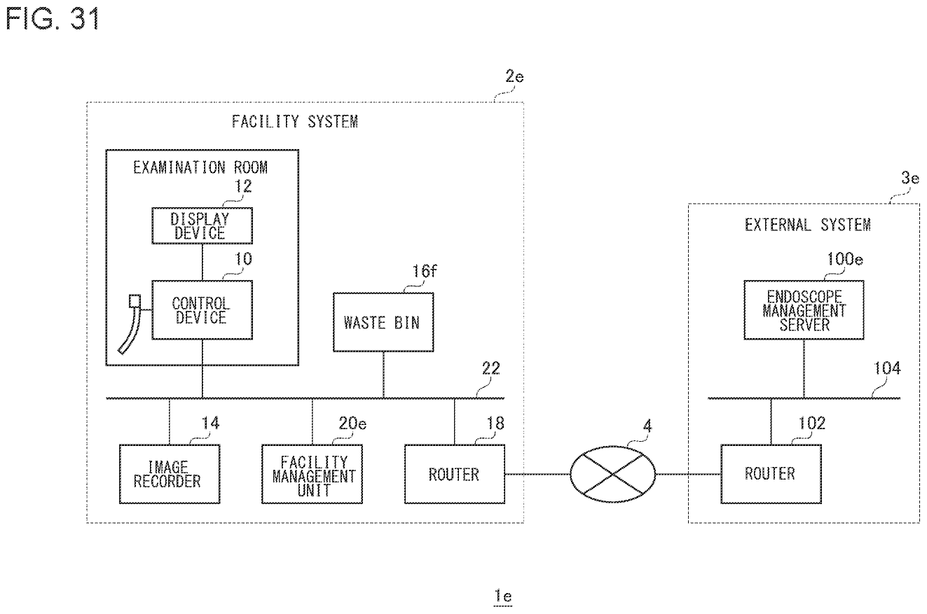

The fifth embodiment discloses a process for managing that an endoscope disposed of has been collected. FIG. 31 shows the configuration of a disposal management system 1e in the fifth embodiment. The disposal management system 1e manages the disposal status of a single-use endoscope in a medical facility and also manages the collection status of the single-use endoscope. The disposal management system 1e includes a facility system 2e, which is installed inside the medical facility, and an external system 3e, which is installed outside the medical facility. The facility system 2e and the external system 3e are communicably connected via the Internet 4. The external system 3e may be operated by a medical device manufacturer that provides the endoscope to the medical facility.

The facility system 2e includes a control device 10, an image recorder 14, a waste bin 16f, and a facility management unit 20e. The control device 10, the image recorder 14, the waste bin 16f, and the facility management unit 20e are communicably connected by a network 22 such as a local area network (LAN). The network 22 is connected to the Internet 4 via a router 18.

The facility management unit 20e manages a work schedule in the medical facility. In the same way as in the facility management unit 20a in the first embodiment, the facility management unit 20e in the fifth embodiment sets an examination schedule in which the dates of multiple endoscopic examinations are set based on multiple examination orders issued by the ordering system and assigns endoscopes that are available at the medical facility to the multiple endoscopic examinations included in the examination schedule. Upon assigning a single-use endoscope to an examination included in the examination schedule and determining a scheduled date (examination date) on which the single-use endoscope is to be used, the facility management unit 20e transmits the scope ID of the single-use endoscope and scheduled date of use information indicating the determined scheduled date of use to the endoscope management server 100e.

The waste bin 16f has a box body in which an endoscope used in an examination (used endoscope) is disposed of and is installed in the medical facility. The waste bin 16f in the fifth embodiment is formed having the same or similar functions as those of the waste bin 16a in the first embodiment.

The external system 3e includes an endoscope management server 100e that manages the disposal status of an endoscope and manages the collection result of the endoscope. The endoscope management server 100e becomes connected to a network 104 such as a local area network (LAN) and becomes connected to the Internet 4 via the router 102. The external system 3d may be operated by a medical device manufacturer that provides the endoscope and the waste bin to the medical facility.

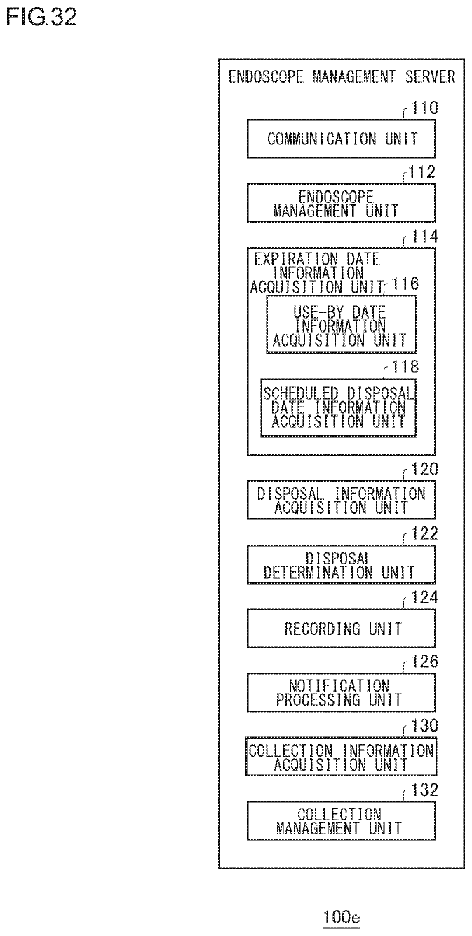

FIG. 32 shows functional blocks of the endoscope management server 100e. The endoscope management server 100e includes a communication unit 110, an endoscope management unit 112, an expiration date information acquisition unit 114, a disposal information acquisition unit 120, a disposal determination unit 122, a recording unit 124, a notification processing unit 126, a collection information acquisition unit 130, and a collection management unit 132. The expiration date information acquisition unit 114 has a function of acquiring scheduled disposal date information and/or use-by date information of an endoscope. In the fifth embodiment, the expiration date information acquisition unit 114 is formed having a use-by date information acquisition unit 116 and a scheduled disposal date information acquisition unit 118.

The configuration shown in FIG. 32 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The endoscope management server 100e has a configuration obtained by adding the collection information acquisition unit 130 that has a function of acquiring collection information indicating that an endoscope disposed of in the medical facility has been collected and the collection management unit 132 that determines whether or not the endoscope disposed of has been actually collected based on the collection information to the configuration of the endoscope management server 100a in the first embodiment. The endoscope management unit 112 generates a management list and records the management list in the recording unit 124, as shown in FIG. 9. Referring to FIG. 9, endoscopes with scope IDs “1001” and “1004” have been disposed of on 2021 Oct. 31.

When the endoscope management unit 112 registers the disposal date in the management list, the collection management unit 132 sets the scheduled collection date for the endoscopes that have been disposed of and registers the scheduled collection date in the management list. The collection management unit 132 may set the scheduled collection date for the endoscopes according to an algorithm for setting the scheduled collection date for the waste bin explained in the fourth embodiment. Alternatively, the collection management unit 132 may set the scheduled collection date for the endoscopes separately from the algorithm explained in the fourth embodiment. The collection management unit 132 sets the scheduled collection date for the endoscopes that have been disposed of in consideration of the availability of the collector and the like.

The collector goes to the medical facility on the scheduled collection date and collects the endoscopes that have been disposed of. The collector reads the respective scope IDs of the collected endoscopes and provides the scope IDs to the endoscope management server 100e. In the endoscope management server 100e, the collection information acquisition unit 130 acquires the scope IDs of the collected endoscopes as the collection information. The collection information acquisition unit 130 provides the acquired scope IDs to the collection management unit 132 along with the acquisition date information indicating the acquisition date. The collection management unit 132 registers a collection confirmation date in the management list in association with the scope IDs of the endoscopes confirmed to have been collected.

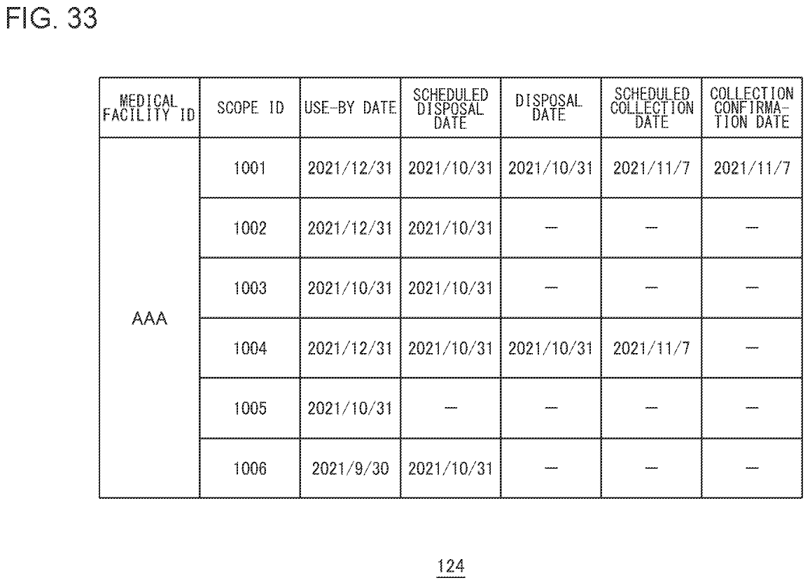

FIG. 33 shows an example of a management list with a field for a scheduled collection date and a field for a collection confirmation date. While the collection management unit 132 determines that the endoscope with the scope ID “1001” has been collected as scheduled, the collection management unit 132 determines that the endoscope with the scope ID “1004” has not been collected as scheduled. Therefore, the notification processing unit 126 may notify the medical facility that the endoscope with the scope ID “1004” has not been collected. By receiving this notification, the medical worker at the medical facility can have an opportunity to check whether a problem has occurred in one of the systems.

Described above is an explanation on the present disclosure based on the embodiments and the exemplary variations. These embodiments and exemplary variations are intended to be illustrative only, and it will be obvious to those skilled in the art that various modifications to constituting elements and processes could be developed and that other modifications are also within the scope of the present disclosure. In the embodiments, it is assumed that the medical device is a single-use medical device. Alternatively, the medical device may be a reusable medical device for loan, which is loaned from a manufacturing facility to a medical facility and returned to the manufacturing facility after being used in the medical facility.

The endoscope management unit 112 or 212 may perform a process of managing the disposal status of endoscopes and replenishing endoscope stocks that are expected to be in short supply at the medical facility. At this time, by managing the types of endoscopes that have been disposed of, the process of replenishing the endoscope stocks for types that are expected to be in short supply at the medical facility can be performed. With an algorithm for performing the replenishment process when a predetermined number of endoscopes are disposed of, the replenishment conditions may be set for each medical facility.

When an endoscope with an expired use-by date is disposed of, the notification processing unit 126 or 226 may notify the medical worker that the use-by date of the endoscope has already expired.

In the third embodiment, it has been explained that the control device 10 determines whether or not to restrict the function of an endoscope based on the use-by date of the endoscope. For an endoscope with a scheduled date of use (scheduled disposal date) that has passed but have enough time until the use-by date, the control device 10 does not need to restrict the function of the endoscope. At this time, the control device 10 may notify the medical worker of a message indicating that the use-by date has expired. On the other hand, for an endoscope with a scheduled date of use (scheduled disposal date) that has passed and without enough time until the use-by date (for example, the use-by date is the next day), the control device 10 may restrict the function of endoscope. Furthermore, for the purpose of strictly preventing reuse, the control device 10 may restrict the function of an endoscope whose scheduled date of use has passed. The medical facility may be able to select when to restrict the function of the endoscope by the control device 10 from these options.

By managing the disposal status of endoscopes, the medical device manufacturer can recognize medical facilities that properly dispose of endoscopes and medical facilities that do not properly dispose of endoscopes. For example, to medical facilities that properly dispose of endoscopes, the medical device manufacturer may give incentives for disposing of endoscopes as scheduled such as lowering the price of the endoscopes the medical device manufacturer provides.

What is claimed is:

1. A disposal management system comprising:
a waste bin in which a medical device is disposed of and one or more processors comprising hardware; wherein
the one or more processors are configured to:
manage identification information of the waste bin and identification information and a disposal date of the medical device that has been disposed of in the waste bin in association with each other; and
set a scheduled date for collecting the waste bin based on at least either one of the number of medical devices that have been disposed of in the waste bin and the disposal date.

2. The disposal management system according to claim 1, wherein the medical device is an endoscope, and
the one or more processors are configured to:
refer to the type of the endoscope that has been disposed of in the waste bin when setting the scheduled date for collecting the waste bin based on the disposal date.

3. The disposal management system according to claim 1, wherein
the one or more processors are configured to:
refer to the type of the waste bin when setting the scheduled date for collecting the waste bin based on the disposal date.

4. The disposal management system according to claim 3, wherein
the waste bin includes a type of waste bin in which the medical device put in a wrapping bag or a wrapping container is inserted and a type of waste bin in which the medical device is directly inserted.

5. The disposal management system according to claim 3, wherein
the waste bin includes a type of waste bin in which the entire box body thereof is collected and a type of waste bin in which a collection bag or a collection container installed inside the box body is collected.

6. The disposal management system according to claim 4, wherein
the one or more processors are configured to:
when the waste bin is the type of waste bin in which the medical device put in a wrapping bag or a wrapping container is inserted, set a scheduled date for supplying wrapping bags or wrapping containers to be the same day as the scheduled collection date for the waste bin or a day prior to the date on which the wrapping bags or the wrapping containers run out based on the remaining amount of wrapping bags or wrapping containers.

7. The disposal management system according to claim 1, wherein
the one or more processors are configured to:
refer to temperature information of an environment in which the waste bin is located, when setting the scheduled date for collecting the waste bin based on the disposal date.

8. The disposal management system according to claim 7, wherein
the temperature information represents the average temperature for a predetermined period in the environment.

9. The disposal management system according to claim 1, wherein
the one or more processors are configured to:
acquire bring-in information indicating that the medical device has been brought in to a disposal site and disposal information indicating that the medical device has been disposed of in the waste bin; and
register the disposal date of the medical device when acquiring both the bring-in information and the disposal information.

10. The disposal management system according to claim 9, wherein
the waste bin becomes to be in a state where the medical device can be disposed of when acquiring the bring-in information.

11. The disposal management system according to claim 1, wherein
the one or more processors are configured to:
set a scheduled supply date for a new waste bin in reference to a scheduled date of use of the medical device set in a work schedule at a medical facility.

12. The disposal management system according to claim 1, wherein
the medical device is an endoscope, and
the waste bin is placed in at least either one of an endoscopic examination vehicle provided with an endoscopic examination room and an endoscope collection vehicle for collecting the endoscope used in the endoscopic examination vehicle.

13. A disposal management system comprising:
a waste bin in which a medical device is disposed of and one or more processors comprising hardware; wherein
the one or more processors are configured to:
manage identification information of the waste bin and identification information and a disposal date of the medical device that has been disposed of in the waste bin in association with each other; and
set a collection schedule for the waste bin in reference to the type of the waste bin.

14. The disposal management system according to claim 1, wherein
the one or more processors are configured to:
acquire scheduled disposal date information or use-by date information of the medical device associated with the identification information of the medical device;
acquire disposal information indicating that the medical device having the identification information has been disposed of; and
determine whether or not the medical device has been disposed of by a scheduled disposal date or a use-by date based on the disposal information.

15. The disposal management system according to claim 14, wherein
the one or more processors are configured to:
hold the scheduled disposal date information or the use-by date information of the medical device in association with the identification information of the medical device; and
determine that the medical device has not been disposed of by the scheduled disposal date or the use-by date if the disposal information has not been acquired by the scheduled disposal date indicated by the scheduled disposal date information or the use-by date indicated by the use-by date information.

16. The disposal management system according to claim 14, wherein
at least one of the processors is provided in a server capable of communicating with the function of acquiring the disposal information via the Internet.

17. A management server that manages a waste bin in which a medical device is disposed of, comprising one or more processors comprising hardware, wherein
the one or more processors are configured to:
manage identification information of the waste bin and identification information and a disposal date of the medical device that has been disposed of in the waste bin in association with each other; and
set a scheduled date for collecting the waste bin based on at least either one of the number of medical devices that have been disposed of in the waste bin and the disposal date.

18. A waste bin management method for managing a waste bin in which a medical device is disposed of, comprising:
acquiring identification information and a disposal date of the medical device that has been disposed of in the waste bin;
managing identification information of the waste bin and the identification information and the disposal date of the medical device that has been disposed of in the waste bin in association with each other; and
setting a scheduled date for collecting the waste bin based on at least either one of the number of medical devices that have been disposed of in the waste bin and the disposal date.
